(19) United States
(12) Patent Application Publication
(10) Pub. No.: US 2010/0274224 A1
(43) Pub. Date: Oct. 28, 2010

(54) LACRIMAL IMPLANT BODY INCLUDING COMFORTING AGENT

(75) Inventors: Rachna Jain, Milpitas, CA (US); Robert W. Shimizu, Laguna Niguel, CA (US); Alan R. Rapacki, Redwood City, CA (US); Sylvie Sim, Omaha, NE (US)

Correspondence Address: Pillsbury Winthrop Shaw Pittman LLP (QLT), P.O. Box 10500, McLean, VA, 22102, US

(73) Assignee: QLT Plug Delivery, Inc., Menlo Park, CA (US)

(21) Appl. No.: 12/499,605

(22) Filed: Jul. 8, 2009

Related U.S. Application Data
(60) Provisional application No. 61/134,271, filed on Jul. 8, 2008.

(51) Int. Cl. A61F 9/00

(52) U.S. Cl. 604/514; 604/285; 264/319

(57) ABSTRACT

Lacrimal implants for treating ocular diseases are disclosed. More particularly, lacrimal punctal plugs, methods of making such plugs, and methods of treating ocular diseases using such plugs are disclosed.

### CLAIM OF PRIORITY

Benefit of priority is hereby claimed to U.S. Provisional Patent Application Ser. No. 61/134,271, filed on Jul. 8, 2008 and entitled Lacrimal Implant Body Including Comforting Agent, the specification of which is herein incorporated by reference in its entirety.

### BACKGROUND

A variety of challenges face patients and physicians in the area of ocular disease management, including drug delivery to the eyes and treatment of dry eyes. Many current ocular drug delivery systems require repetitive administration and are often ineffective due to a lack of patient compliance. Many current tear flow blockage techniques also have drawbacks, including being irreversible in nature.

In order to eye treat infection and inflammation of the eye, glaucoma and other ocular diseases, drugs are often required to be administered to the eye. A conventional method of drug delivery is by topical drop application to the eye's surface. However, such topically applied drugs (e.g., eye drops) suffer from poor patient compliance and untimely application. For instance, when an eye drop is instilled in an eye, it often overfills the conjunctival sac (i.e., the pocket between the eye and the lids) causing a substantial portion of the drop to be lost due to overflow of the lid margin and spillage onto the cheek. Large portions of the drop remaining on the ocular surface can be washed away into and through a lacrimal canaliculus, thereby diluting the concentration of the drug before it can treat the eye. Moreover, topically applied drugs often have a peak ocular effect for about two hours post-application, after which additional applications of the drugs should be, but are often not, administered to maintain the desired therapeutic benefit.

To compound ocular management difficulty, patients often do not use their eye drops as prescribed. This poor compliance can be due to, for example, an initial stinging or burning sensation caused by the eye drop and experience by a patient. Instilling eye drops in one's own eye can be difficult, in part because of the normal reflex to protect the eye. Therefore, one or more drops may miss the eye. Older patients may have additional problems instilling drops due to arthritis, unsteadiness, and decreased vision. Pediatric and psychiatric populations pose difficulties as well.

Conditions of dry eye have been treated by blocking the tear flow from the eye into and through the lacrimal canaliculus. This has involved closing the canalicular canal by stitching the punctal opening shut or by using electrical or laser cauterization to seal the punctal opening. Although such procedures can provide the desired result of blocking tear flow to treat a dry eye, they are unfortunately not reversible without reconstructive surgery.

In light of the above, what is needed are improved drug delivery systems that overcome at least some of the above shortcomings.

### SUMMARY

The present subject matter pertains generally to lacrimal implants. More particularly, this present subject matter pertains to lacrimal punctal plugs, methods of making such plugs, and methods of treating ocular diseases using such plugs.

In some embodiments, a punctum plug that is insertable into a lacrimal punctum for release of a therapeutic agent or agents, a comforting agent or agents, or both to an eye is provided. The punctum plug includes a plug body having a proximal end adapted to be disposed near the punctal opening and a distal end adapted to be disposed within the punctal canal. In some embodiments, the plug body has an optional agent core at or near the proximal end portion and a retention structure at or near the distal end portion. The agent core or the plug body can have at least one surface that provides release of the therapeutic agent or agents, the comforting agent or agents, or both, to the eye. In some embodiments, the agent core provides release of the therapeutic agent or agents. In other embodiments, the plug body provides release of the therapeutic agent or agents. In other embodiments, both the agent core and the plug body provide release of the therapeutic agent or agents. Similarly, in some embodiments, the agent core provides release of the comforting agent or agents. In other embodiments, the plug body provides release of the comforting agent or agents. In other embodiments, both the agent core and the plug body provide release of the comforting agent or agents. Thus, it is contemplated that the agent core can provide release of both the therapeutic agent or agents and the comforting agent or agents. Likewise, the plug body can provide release of both the therapeutic agent or agents and the comforting agent or agents.

The comforting agent or agents can be, but are not limited to, a demulcent or emollient. Comforting agents for use in the punctum plugs described herein include, but are not limited to, polyvinyl alcohol (PVA), polyethylene oxide (PEO), polyvinyl pyrrolidone (PVP), hydroxypropyl methylcellulose (HPMC), carboxy methylcellulose (CMC), glycerin, other compounds listed in 21 CFR, such as at §§349.12 and 349.14, simethicone, and sodium hyaluronate. The release of the therapeutic agent can be sustained release, while the comforting agent can be released for up to about two weeks, up to about four weeks, up to about six weeks, or up to about eight weeks. In some embodiments, the release of the comforting agent is more than eight weeks.

In some embodiments, the plug body includes silicone, a silicone copolymer, a polyurethane, a polyurethane copolymer, a polyurethane-silicone copolymer, or combinations thereof. The composition of the plug body can control the release rate of the comforting agent or therapeutic agent.

The comforting agent or agents and/or therapeutic agent or agents can be coated on the surface of the plug body, agent core, or both. The comforting agent or agents and/or therapeutic agent or agents can also be infused into the plug body, agent core, or both. In some embodiments, the comforting agent or agents and/or therapeutic agent or agents are mixed into the plug body, agent core, or both. The comforting agent or agents and/or therapeutic agent or agents can be embedded in the plug body.

In certain embodiments, the therapeutic agent is an anti-glaucoma medication. Anti-glaucoma medications contemplated for use in the punctum plugs described herein include, but are not limited to, adrenergic agonists, adrenergic antagonists, carbonic anhydrase inhibitors, parasympathomimetics, prostaglandins and hypotensive lipids, and combinations thereof. In certain embodiments, the anti-glaucoma medication is latanoprost. In other embodiments, the therapeutic agent is cyclosporine. In other embodiments, the therapeutic agent is a corticosteroid or other anti-inflammatory, an agent that prevents of modifies an allergic response, or a mast cell stabilizer.

In certain punctum plugs, the agent core has at least one therapeutic agent inclusion distributed in a solid matrix and at least partially covered by a sheath body to define at least one exposed agent core surface. The solid matrix can be a mixture of silicone and the therapeutic agent, or can be a mixture of silicone, the therapeutic agent, and the comforting agent.

The punctum plugs described herein can be useful for the treatment of dry eye. In some embodiments, the treatment of dry eye further includes the use of cyclosporine drops. In other embodiments, the treatment of dry eye includes the release of both cyclosporine and a comforting agent from the punctum plug.

Also contemplated herein are kits for treating an eye disease, including the punctum plugs described herein and instructions for use. In some embodiments, the punctum plug is individually packaged for a single use. In some embodiments glaucoma, pre- and post-surgical ocular conditions, dry eye, eye infections, post-surgical inflammation or pain, allergies, or inner ear disorders, such as dizziness or migraines, can be treated using the described kits.

Also provided are methods of manufacturing the punctum plugs insertable into a lacrimal punctum for release of a therapeutic agent or agents and a comforting agent or agents to an eye. In certain embodiments, a plug body is formed having a proximal end adapted to be disposed near the punctal opening and a distal end adapted to be disposed within the punctal canal. The distal end is manufactured to have a retention structure located at or near to the distal end. An optional preformed agent core having at least one surface providing release of an agent to the eye can be inserted into the plug body at or near the proximal end of the plug body. The agent core, plug body, or both can provide release of the comforting agent or agents to the eye. In some embodiments, the plug body is formed by mixing the comforting agent into a silicone material; placing the mixture into a mold; and heat curing the mixture. In some embodiments, the plug body is formed by infusing the comforting agent or agents into a silicone material during a curing process.

Methods to treat a subject having an eye disorder are also provided. In some embodiments, glaucoma, pre- and post-surgical ocular conditions, dry eye, eye infections, post-surgical inflammation or pain, allergies, or inner ear disorders, such as dizziness or migraines, and/or ocular hypertension are treated. Adrenergic agonists, adrenergic antagonists, carbonic anhydrase inhibitors, parasympathomimetics, prostaglandins and hypotensive lipids, and combinations thereof are contemplated for use in the punctum plugs described herein. In some embodiments, cyclosporine or latanoprost drops are administered in conjunction with the use of the punctum plugs.

In some embodiments, a punctum plug as described above is inserted into at least one lacrimal punctum of the subject to provide a comforting agent, a therapeutic agent, or both, to the eye. The punctum plug can have a plug body having a proximal end adapted to be disposed near the punctal opening and a distal end adapted to be disposed within the punctal canal. The plug body can include an optional agent core at or near the proximal end portion and a retention structure at or near the distal end portion. The agent core can include the comforting agent or agents and/or therapeutic agent or agents and has at least one surface providing release of the agent to the eye. The comforting agent can be a demulcent or emollient. In some embodiments, a subject treated with a punctum plug including a comforting agent may experience less discomfort compared to a subject who is treated with a punctum plug lacking the comforting agent.

In certain embodiments, the inserted punctum plug is replaced with a second punctum plug having the same, lower or higher dosage of the agent following an interval of time. In some embodiments, the interval of time is at least one month. In some embodiments, replacing the punctum plug is repeated at least two, three, four or five times. Replacing the punctum plug can be repeated until the subject no longer requires treatment. The comforting agent or agents can be released from the plug for at least one week, at least two weeks, or at least one month, while the therapeutic agent or agents can be released for up to 90 days.

Also contemplated herein is a punctum plug insertable into a lacrimal punctum, including a plug body having a proximal end adapted to be disposed near the punctal opening and a distal end adapted to be disposed within the punctal canal. The punctum plug provides release of an agent providing lubrication, comfort, hydration, or combinations thereof.

### BRIEF DESCRIPTION OF THE FIGURES

In the drawings, like numerals can be used to describe similar components throughout the several views. The drawings illustrate generally, by way of example, but not by way of limitation, various embodiments discussed in the present document.

FIG. 1 illustrates an example of a cross-sectional view of a punctum plug configured to be retained at least partially within a lacrimal punctum or canalicular anatomy.

FIG. 2A illustrates an example of an isometric view of a punctum plug configured to be retained at least partially within a lacrimal punctum or canalicular anatomy.

FIG. 2B illustrates an example of a cross-sectional view of a punctum plug taken along a line parallel to a longitudinal axis of the plug, such as along line 2B-2B of FIG. 2A.

FIG. 2C illustrates an example of a cross-sectional view of another punctum plug taken along a line parallel to a longitudinal axis of the plug.

FIG. 3A illustrates an example of an isometric view of a punctum plug configured to be retained at least partially within a lacrimal punctum or canalicular anatomy.

FIG. 3B illustrates an example of a cross-sectional view of a punctum plug taken along a line parallel to a longitudinal axis of the plug, such as along line 3B-3B of FIG. 3A, and a dilation of a plug-receiving anatomical tissue structure.

FIG. 4A illustrates an example of an isometric view of a punctum plug configured to be retained at least partially within a lacrimal punctum or canalicular anatomy.

FIG. 4B illustrates an example of a cross-sectional view of a punctum plug taken along a line parallel to a longitudinal axis of the plug, such as along line 4B-4B of FIG. 4A.

### DETAILED DESCRIPTION

### Definitions

As used herein, the terms “a” or “an” are used, as is common in patent documents, to include one or more than one, independent of any other instances or usages of “at least one” or “one or more.”

As used herein, the term “and/or” is meant to be both inclusive and exclusive, such that “A and/or B” includes “A but not B,” “B but not A,” and “A and B,” unless otherwise indicated.

As used herein, the term “about” is used to refer to an amount that is approximately, nearly, almost, or in the vicinity of being equal to a stated amount. The terms “about” and “approximately” are used interchangeably throughout this document.

The term “comforting agent” as used herein refers to an agent that comforts, soothes, provides hydration or lubrication, and/or relieves ocular irritation associated with topical administration of certain therapeutic agents and/or the use of punctum plugs. Comforting agents include, but are not limited to, demulcents and emollients. A demulcent is an agent, usually a water-soluble polymer, which can be applied topically to the eye to protect and lubricate mucous membrane surfaces and relieve dryness and irritation. An emollient is an agent, usually a fat or oil, which can be applied locally to the eye to protect or soften tissues and to prevent drying and cracking. As used herein, “a comforting agent” can refer to a single comforting agent or a combination of more than one comforting agent.

As used herein, the phrase “consisting essentially of” limits a composition to the specified materials or steps and those additional, undefined components that do not materially affect the basic and novel characteristic(s) of the composition.

As used herein, the term “continuous” or “continuously” means unbroken or uninterrupted. For example, continuously administered therapeutic or comforting agents are administered over a period of time without interruption.

As used herein, the term “eye” refers to any and all anatomical tissues and structures associated with an eye. The eye is a spherical structure with a wall having three layers: the outer sclera, the middle choroid layer and the inner retina. The sclera includes a tough fibrous coating that protects the inner layers. It is mostly white except for the transparent area at the front, the cornea, which allows light to enter the eye. The choroid layer, situated inside the sclera, contains many blood vessels and is modified at the front of the eye as the pigmented iris. The biconvex lens is situated just behind the pupil. The chamber behind the lens is filled with vitreous humour, a gelatinous substance. The anterior and posterior chambers are situated between the cornea and iris, respectively and filled with aqueous humour. At the back of the eye is the light-detecting retina. The cornea is an optically transparent tissue that conveys images to the back of the eye. It includes avascular tissue to which nutrients and oxygen are supplied via bathing with lacrimal fluid and aqueous humour as well as from blood vessels that line the junction between the cornea and sclera. The cornea includes one pathway fro the permeation of drugs into the eye. Other anatomical tissue structures associated with the eye include the lacrimal drainage system, which includes a secretory system, a distributive system and an excretory system. The secretory system comprises secretors that are stimulated by blinking and temperature change due to tear evaporation and reflex secretors that have an efferent parasympathetic nerve supply and secrete tears in response to physical or emotional stimulation. The distributive system includes the eyelids and the tear meniscus around the lid edges of an open eye, which spread tears over the ocular surface by blinking, thus reducing dry areas from developing.

A ‘hydrogel” as the term is used herein refers to a polymeric material that has absorbed greater than 100 wt %, for example up to 500-2000 wt %, of water within the polymeric structure and has consequently swelled substantially in physical size. A hydrogel possesses physical integrity, has tensile strength, and is not substantially fluid. A “hydrogel-forming polymer” is a polymeric material capable of forming a hydrogel upon contact with water.

As used herein, the term “implant” refers to a structure that can be configured to contain or be impregnated with a core or a matrix, such as those as disclosed in this patent document and in WO 07/115,261, which is herein incorporated by reference in its entirety, which is capable of releasing a quantity of a therapeutic and/or comforting agent or agents into tear fluid for a sustained release period of time when the structure is implanted at a target location along the path of the tear fluid in the patient. The terms “implant,” “plug” and “punctum plug” are meant herein to refer to similar structures. Likewise, the terms “implant body” and “plug body” are meant herein to refer to similar structures. The implants described herein may be inserted into the punctum of a subject, or through the punctum into the canaliculus. The implant may be also a core or therapeutic and/or comforting agent matrix itself, which is configured for insertion into the punctum without being housed in a carrier such as a punctal plug occluder, for example having a polymeric component and a therapeutic and/or comforting agent component with no additional structure surrounding the polymeric component and therapeutic agent component.

As used herein, a “pharmaceutically acceptable vehicle” is any physiological vehicle known to those of ordinary skill in the art useful in formulating pharmaceutical compositions. Suitable vehicles include polymeric matrices, sterile distilled or purified water, isotonic solutions such as isotonic sodium chloride or boric acid solutions, phosphate buffered saline (PBS), propylene glycol and butylene glycol. Other suitable vehicular constituents include phenylmercuric nitrate, sodium sulfate, sodium sulfite, sodium phosphate and monosodium phosphate. Additional examples of other suitable vehicle ingredients include alcohols, fats and oils, polymers, surfactants, fatty acids, silicone oils, humectants, moisturizers, viscosity modifiers, emulsifiers and stabilizers. The compositions may also contain auxiliary substances, i.e. antimicrobial agents such as chlorobutanol, parabans or organic mercurial compounds; pH adjusting agents such as sodium hydroxide, hydrochloric acid or sulfuric acid; and viscosity increasing agents such as methylcellulose. The final composition should be sterile, essentially free of foreign particles, and have a pH that allows for optimum drug stability.

A “polymer” as the term is used herein, refers to an organic macromolecule containing one or more repeating units. A “copolymer” refers to a polymer in which there are at least two types of repeating units included. A copolymer can be a block copolymer, in which there are segments containing multiple repeating units of one type, bonded to segments containing multiple repeating units of a second type.

A “polyurethane” refers to a variety of polymer or copolymer containing repeating units bonded covalently through urethane, i.e., carbamate, bonds, —N—C(O)—O— wherein the N and O atoms are attached to an organic radical. The organic radical can be aliphatic, aromatic, or mixed; can contain other functional groups. Each radical, other than the radicals at the ends of the molecular chains, is bonded via two (or more) urethane groups to other radicals. A polyurethane polymer contains only urethane-type groups joining the repeating units. A polyurethane copolymer, such as a polyurethane-silicone copolymer or a polyurethane-carbonate copolymer, contains urethane and other types of groups joining the repeating units, i.e., silicone and carbonate type groups respectively. Examples include Elast-Eon™ by AorTech, a polyurethane-silicone copolymer, Tecoflex® by Lubrizol, an aliphatic flexible polyurethane, Tecothane® by Lubrizol, a thermoplastic polyurethane, and Carbothane® by Lubrizol, a polyurethane/polycarbonate copolymer.

A polyurethane-silicone copolymer contains segments of polyurethane chains and segments of silicone chains. An example of a polyurethane-silicone copolymer is “Pursil®”, a product of Polymer Technologies Inc., of Berkeley, Calif., described by the manufacturer as a family of aliphatic or aromatic, thermoplastic silicone polyether urethane copolymers. These polymers are formed by the incorporation of silicone in the polymer backbone together with polyether soft segments, and the use of Surface-Modifying End Groups™ (SME) to terminate the polymer chain. Aromatic silicone polyether urethane copolymers are also useful in the punctum plugs described herein. A polyurethane-carbonate copolymer contains urethane segments and carbonate (—O—C(O)O—) segments. An example of a polyurethane-carbonate copolymer is Carbothane TPU® (Lubrizol).

As used herein, the term “punctum” refers to the orifice at the terminus of the lacrimal canaliculus, seen on the margins of the eyelids at the lateral extremity of the lacus lacrimalis. Puncta (plural of punctum) function to reabsorb tears produced by the lacrimal glands. The excretory part of the lacrimal drainage system includes, in flow order of drainage, the lacrimal puncta, the lacrimal canaliculi, the lacrimal sac and the lacrimal duct. From the lacrimal duct, tears and other flowable materials drain into a passage of the nasal system. The lacrimal canaliculi include an upper (superior) lacrimal canaliculus and a lower (inferior) lacrimal canaliculus, which respectively terminate in an upper and lower lacrimal punctum. The upper and lower punctum are slightly elevated at the medial end of a lid margin at the junction of the ciliary and lacrimal portions near a conjunctival sac. The upper and lower punctum are generally round or slightly ovoid openings surrounded by a connective ring of tissue. Each of the puncta leads into a vertical portion of their respective canaliculus before turning more horizontal at a canaliculus curvature to join one another at the entrance of the lacrimal sac. The canaliculi are generally tubular in shape and lined by stratified squamous epithelium surrounded by elastic tissue, which permits them to be dilated.

The terms “subject” and “patient” refer to animals such as mammals, including, but not limited to, primates (e.g., humans), cows, sheep, goats, horses, dogs, cats, rabbits, rats, mice and the like. In many embodiments, the subject or patient is a human.

A “therapeutic agent” can comprise a drug and may be any of the following or their equivalents, derivatives or analogs, including anti-glaucoma medications (e.g. adrenergic agonists, adrenergic antagonists (beta blockers), carbonic anhydrase inhibitors (CAIS, systemic and topical), parasympathomimetics, prostaglandins and hypotensive lipids, and combinations thereof), antimicrobial agents (e.g., antibiotic, antiviral, antiparacytic, antifungal, etc.), a corticosteroid or other anti-inflammatory (e.g., an NSAID or cyclosporine), a decongestant (e.g., vasoconstrictor), an agent that prevents of modifies an allergic response (e.g., an antihistamine, cytokine inhibitor, leucotriene inhibitor, IgE inhibitor, immunomodulator, immunosuppressant), a mast cell stabilizer, cycloplegic or the like. Examples of conditions that may be treated with the therapeutic agent(s) include but are not limited to glaucoma, pre- and post-surgical ocular conditions, dry eye, eye infections, post-surgical inflammation or pain, allergies, or inner ear disorders, such as dizziness or migraines.

Exemplary therapeutic agents include, but are not limited to thrombin inhibitors; antithrombogenic agents; thrombolytic agents; fibrinolytic agents; vasospasm inhibitors; vasodilators; antihypertensive agents; antimicrobial agents, such as antibiotics (such as tetracycline, chlortetracycline, bacitracin, neomycin, polymyxin, gramicidin, cephalexin, oxytetracycline, chloramphenicol, rifampicin, ciprofloxacin, tobramycin, gentamycin, erythromycin, penicillin, sulfonamides, sulfadiazine, sulfacetamide, sulfamethizole, sulfisoxazole, nitrofurazone, sodium propionate), antifungals (such as amphotericin B and miconazole), and antivirals (such as idoxuridine trifluorothymidine, acyclovir, gancyclovir, interferon); inhibitors of surface glycoprotein receptors; antiplatelet agents; antimitotics; microtubule inhibitors; anti-secretory agents; active inhibitors; remodeling inhibitors; antisense nucleotides; anti-metabolites; antiproliferatives (including antiangiogenesis agents); anticancer chemotherapeutic agents; anti-inflaTnmatories (such as hydrocortisone, hydrocortisone acetate, dexamethasone 21-phosphate, fluocinolone, medrysone, methylprednisolone, prednisolone 21-phosphate, prednisolone acetate, fluoromethalone, betamethasone, triamcinolone, triamcinolone acetonide); non steroidal anti-inflammatories (NSAIDs, such as salicylate, indomethacin, ibuprofen, diclofenac, flurbiprofen, piroxicam indomethacin, ibuprofen, naproxen, ketorolac, piroxicam and nabumetone). Such anti inflammatory steroids contemplated for use in the methodology of the present subject matter, include triamcinolone acetonide (generic name) and corticosteroids that include, for example, triamcinolone, dexamethasone, fluocinolone, cortisone, prednisolone, flumetholone, and derivatives thereof.); antiallergenics (such as sodium chromoglycate, antazoline, methapyriline, chlorpheniramine, cetrizine, pyrilamine, prophenpyridamine, loteprednol, epinastine, emedastine, levocabastine, azelastine, olopatadine, ketotifen, ketorolac, lodoxamide, cromolyn, pemirolost, and nedocromil); anti proliferative agents (such as 1,3-cis retinoic acid, 5-fluorouracil, taxol, rapamycin, mitomycin C and cisplatin); decongestants (such as phenylephrine, naphazoline, tetrahydrazoline); miotics and anti-cholinesterase (such as pilocarpine, salicylate, carbachol, acetylcholine chloride, physostigmine, eserine, diisopropyl fluorophosphate, phospholine iodine, demecarium bromide); antineoplastics (such as carmustine, cisplatin, fluorouracil3; immunological drugs (such as vaccines and immune stimulants); hormonal agents (such as estrogens, -estradiol, progestational, progesterone, insulin, calcitonin, parathyroid hormone, peptide and vasopressin hypothalamus releasing factor); immunosuppressive agents, growth hormone antagonists, growth factors (such as epidermal growth factor, fibroblast growth factor, platelet derived growth factor, transforming growth factor beta, somatotrapin, fibronectin); inhibitors of angiogenesis (such as angiostatin, anecortave acetate, thrombospondin, anti-VEGF antibody); dopamine agonists; radiotherapeutic agents; peptides; proteins; enzymes; extracellular matrix; components; ACE inhibitors; free radical scavengers; chelators; antioxidants; anti polymerases; photodynamic therapy agents; gene therapy agents; and other therapeutic agents such as prostaglandins, antiprostaglandins, prostaglandin precursors, including antiglaucoma drugs including beta-blockers such as Timolol, betaxolol, levobunolol, atenolol, and prostaglandin analogues such as Bimatoprost, travoprost, Latanoprost etc; carbonic anhydrase inhibitors such as acetazolamide, dorzolamide, brinzolamide, methazolamide, dichlorphenamide, diamox; and neuroprotectants such as lubezole, nimodipine and related compounds; and parasympathomimetrics such as pilocarpine, carbachol, physostigmine and the like.

The term “topical” refers to any surface of a body tissue or organ. A topical formulation is one that is applied to a body surface, such as an eye, to treat that surface or organ. Topical formulations include liquid drops such as eye drops; creams, lotions, sprays, emulsions, and gels. Topical formulations as used herein also include formulations that release therapeutic agents into the tears to result in topical administration to the eye.

As used herein, the term “treating” or “treatment” of a disease includes: (1) preventing the disease, i.e., causing the clinical symptoms of the disease not to develop in a subject that may be exposed to or predisposed to the disease but who does not yet experience or display symptoms of the disease; (2) inhibiting the disease, i.e., arresting or reducing the development of the disease or its clinical symptoms; or (3) relieving the disease, i.e., causing regression of the disease or its clinical symptoms. Examples of diseases or disorders that can be treated with above-listed agents using the inventive punctum plugs include, but are not limited to, glaucoma, pre- and post-surgical ocular conditions, dry eye, eye infections, post-surgical inflammation or pain, allergies, or inner ear disorders, such as dizziness or migraines. Furthermore, inflammatory diseases of the conjunctive can be treated using a present punctum plug comprising a corticosteroid, NSAID, or an agent intended to prevent, inhibit or modify an allergic response, such as an antihistamine, or a mast cell stabilizer.

### Patient Noncompliance:

Numerous studies have been published showing high noncompliance by patients using eye drops for treatment of various ocular disorders. One study showed only 64% of patients used the eye drops as directed (Winfield et al., 1990). Another study showed that 41% of patients using eye drops for glaucoma missed six or more doses over a 30-day period (Norell and Granstrom, 1980). Patients using OTC ocular formulations may use sterile ophthalmic solution drops several times during the day. Therefore, they must carry containers with them; either multi-dose preserved formulations or multiple units of non-preserved unit dose containers. The inconvenience and cost of these therapies can add to the factors that limit the effectiveness of ophthalmic formulations.

The subject matter described herein provides punctum plugs, methods to make the plugs, and methods to treat ocular conditions. The punctum plugs described herein can benefit from one or more of: the ability to be easily implanted and removed without much biasing of the lacrimal punctum or canaliculus, the ability to be securely retainable in the lacrimal punctum upon implantation, the ability to allow for the sustained, localized release of one or more agents (e.g., a comforting agent or agents, a therapeutic agent or agents, or combinations thereof) at desired therapeutic levels, and the ability to avoid the problem of noncompliance often associated with eye drop administration. In some embodiments, the punctum plugs and methods reduce patient noncompliance significantly compared to eye drop administration, by at least 10%, at least 20%, at least 30%, at least 40%, at least 50%, at least 60%, at least 70%, at least 80%, or at least 90%. In some embodiments, overall patient noncompliance with the methods described herein is about 5%, about 10%, about 15%, about 20%, or about 25%.

### Implant:

The implants, also referred to as punctum plugs, as described herein can be configured, when implanted at a target location along the path of tear fluid in the patient, to release a quantity of therapeutic and/or comforting agent into the tear fluid each day for a period of days, weeks, or months. The implant can be one of any number of different designs that releases a therapeutic and/or comforting agent for a period of time. The disclosures of the following patent documents, which describe example implant embodiments for use with the present subject, are incorporated herein by reference in their entirety: U.S. Application Ser. No. 60/871,864 (filed Dec. 26, 2006 and entitled Nasolacrimal Drainage System Implants for Drug Therapy); U.S. application Ser. No. 11/695,537 (filed Apr. 2, 2007 and entitled Drug Delivery Methods, Structures, and Compositions for Nasolacrimal System); U.S. Application Ser. No. 60/787,775 (filed Mar. 31, 2006 and entitled Nasolacrimal drainage system implants for drug therapy); U.S. application Ser. No. 11/695,545 (filed Apr. 2, 2007 and entitled Nasolacrimal drainage system implants for drug therapy); U.S. Application Ser. No. 60/970,696 (filed Sep. 7, 2007 and entitled Expandable Nasolacrimal Drainage System Implants); U.S. Application Ser. No. 60/974,367 (filed Sep. 21, 2007 and entitled Expandable Nasolacrimal Drainage System Implants); U.S. Application Ser. No. 60/970,699 (filed Sep. 7, 2007 and entitled Manufacture of Drug Cores for Sustained Release of Therapeutic Agents); U.S. Application Ser. No. 60/970,709 (filed Sep. 7, 2007 and entitled Nasolacrimal Drainage System Implants for Drug Delivery); U.S. Application Ser. No. 60/970,720 (filed Sep. 7, 2007 and entitled Manufacture of Expandable Nasolacrimal Drainage System Implants); U.S. Application Ser. No. 60/970,755 (filed Sep. 7, 2007 and entitled Prostaglandin Analogues for Implant Devices and Methods); U.S. Application Ser. No. 60/970,820 (filed Sep. 7, 2007 and entitled Multiple Drug Delivery Systems and Combinations of Drugs with Punctal Implants); U.S. Application Ser. No. 61/049,347 (filed Apr. 30, 2008 and entitled Lacrimal Implants and Related Methods); U.S. Application Ser. No. 61/049,360 (filed Apr. 30, 2008 and entitled Lacrimal Implants and Related Methods); U.S. Application Ser. No. 61/036,816 (filed Mar. 14, 2008 and entitled Lacrimal Implants and Related Methods); U.S. Application Ser. No. 61/049,337 (filed Apr. 30, 2008 and entitled Lacrimal Implants and Related Methods); U.S. Application Ser. No. 61/049,329 (filed Apr. 30, 2008 and entitled Composite Lacrimal Insert); U.S. Application Ser. No. 61/049,317 (filed Apr. 30, 2008 and entitled Drug-Releasing Polyurethane Lacrimal Insert); U.S. application Ser. No. 10/825,047 (filed Apr. 15, 2004 and entitled Drug Delivery via Punctal Plug); International Published Application WO 2006/014434; and International Application Serial No. PCT/US2007/065789 (filed Mar. 31, 2006, published as WO 2007/115259 and entitled Nasolacrimal Drainage System Implants for Drug Therapy).

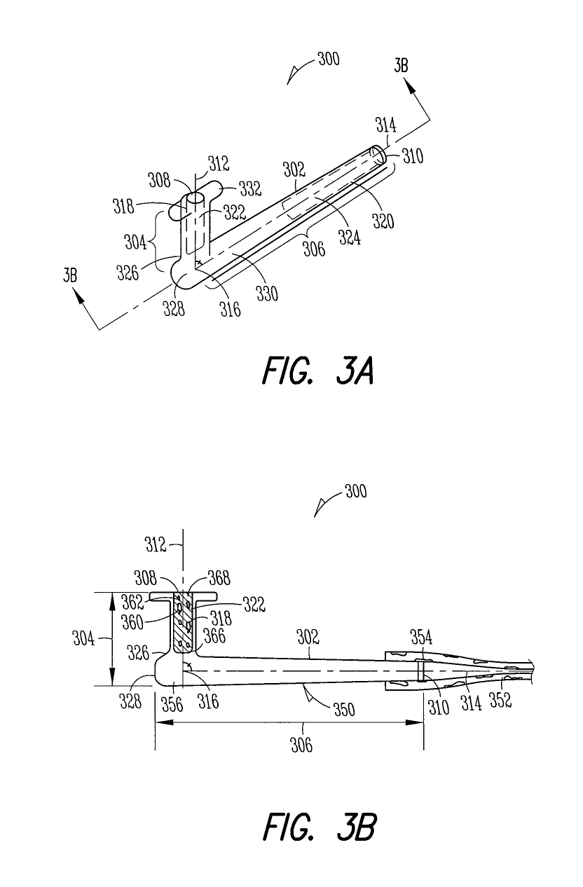

For example, the plug can be substantially cylindrical, or the plug can be of a conical shape, or can be bent in the form of an “L-shape” or “bent” design (see FIGS. 3A and 3B) or can have any other shape which can be disposed within the punctal canal of a patient's eye such that a therapeutic and/or comforting agent that is contained within a portion of the plug, either dispersed in the polymer making up a first plug portion, or with a core insert adapted to be inserted into a receptacle in the first plug portion, can be released into the tear fluid bathing the eye. Accordingly, when the plug is disposed within the punctal canal, the first plug portion can have access to the opening of the punctum such that the therapeutic and/or comforting agent can diffuse into the tear fluid and thereby bathe the eye surface. The plug can also be a design resembling an “H-shape”, wherein swelling and non-swelling polymers are bonded to one another for retention in the punctum.

Generally, the implant comprises a body. In some embodiments, the implant body has a distal end portion and a proximal end portion. The distal end portion of the body is at least partially insertable into the punctum to the canalicular lumen of the patient.

Advantageously, in some examples, the present punctum plugs can successfully block the flow of tears and/or provide delivery of a therapeutic and/or comforting agent to an eye, nasal passage, or inner ear for varying periods of time, such as from days to months to years.

In various embodiments, an implant comprising a polyurethane polymer or copolymer is provided. Typically, punctum plugs are formed of silicone polymers, which can be quite hydrophobic and furthermore are usually prepared by polymerization of a silicone precursor in the presence of a catalyst. However, polyurethane polymers and copolymers can be thermoplastic, and can therefore be melted and cast into a desired form. A therapeutic and/or comforting agent can be dispersed within the polyurethane melt, either in molten form itself or as a dispersion of a solid material. Polyurethane polymers and copolymers can also be dissolved in various organic solvents, such as dichloromethane or tetrahydrofuran, then cast into a desired form with removal of the solvent, such as by evaporation. A therapeutic and/or comforting agent can be dispersed or dissolved in the organic solvent along with the polyurethane, such that upon removal of the solvent, the polyurethane containing the agent in a desired form is obtained.

The implant body may be at least impregnated with a therapeutic and/or comforting agent or otherwise comprise a therapeutic and/or comforting agent, such as within an optional matrix core that is inserted into the implant body. Exposure of the matrix core or impregnated body to tear fluid can cause an effective agent release into the tear fluid. The implant may include a sheath disposed over at least a portion of the optional core to inhibit release of the therapeutic and/or comforting agent from certain portions thereof. The implant body may have an outer surface configured to engage luminal wall tissues so as to inhibit expulsion when disposed therein. In many embodiments, an integral feedback or other projection is connected around the sheath near the proximal end of the optional core. In an embodiment, the feedback or other projection includes one or more wings sized to remain outside the punctum so as to retain the proximal end of the core near the punctum. In other embodiments, the feedback or other projection includes a full or partial (e.g., trimmed) collar connected around the sheath near the proximal end of the core. The collar can be sized to remain outside the punctum so as to retain the proximal end of the core near the punctum.

In some embodiments, the implant comprises a core alone, lacking an additional structure surrounding the core. In some embodiments, the core comprises a matrix comprising a pharmaceutically acceptable vehicle, for example, a non-bioabsorbable polymer, for example silicone in a non-homogenous mixture with a therapeutic and/or comforting agent. The non-homogeneous mixture in the core may comprise a silicone matrix saturated with the therapeutic and/or comforting agent or with inclusions of therapeutic and/or comforting agent. The inclusions in the core are a concentrated form of agent, and the silicone matrix encapsulates the inclusions in the core. In specific embodiments, the agent inclusions encapsulated within the silicone matrix comprise an inhomogeneous mixture of the inclusions encapsulated within the silicone matrix. It is also within the scope of the present subject matter to modify or adapt the implant device to deliver a high release rate, a low release rate, a bolus release, a burst release, or combinations thereof. A bolus of the therapeutic and/or comforting agent may be released by the formation of an erodable polymer cap that is immediately dissolved in the tear or tear film. As the polymer cap comes in contact with the tear or tear film, the solubility properties of the polymer enable the cap to erode and the agent or agents are released at substantially the same time. A burst release of agent can be performed using a polymer that also erodes in the tear or tear film based on the polymer solubility. In an example, the therapeutic and/or comforting agent and polymer may be stratified along the length of the device so that as the outer polymer layer dissolves, the therapeutic and/or comforting agent is immediately released. An initial burst can also be generated by surface coating of the polymer matrix to increase the material available for dissolution immediately upon insertion. A high or low release rate of the therapeutic and/or comforting agent could be accomplished by changing the solubility of the erodable polymer layer so that the agent layer releases quickly or slowly. Other methods to release the agent could be achieved through porous membranes, soluble gels (such as those in typical ophthalmic solutions), microparticle encapsulations of the drug, or nanoparticle encapsulation.

Controlling the rate of release of the comforting agent or agents can also be achieved by modulating polymer size and/or cross-linking of either the matrix polymer (e.g., silicone) or agents (e.g., polymeric demulcents such as CMC, HPMC, PEO, PVA, PVP, etc.). In addition, chemical changes (e.g., percent hydrolysis for PVA) can change the physical chemical properties which alter elution profiles. As an example, degrees of hydrolysis and molecular weight changes of PVA can effect the ability of the molecule to form areas of crystalline and amorphous arrangements that can affect water penetration between molecules altering solubility and ultimately the rate of delivery. Additionally, changes in degrees of hydrolysis and molecular weight of PVA, for example, can alter the material's tensile strength.

### Comforting Agent:

Certain therapeutic agents can irritate the tissues of the eye when administered. Burning and stinging sensations are common side effects of topically-applied medicaments. Additionally, the molding process for punctum plugs can form excess flash material, sharp edges, or other irregularities in a molded article's structure. Punctum plugs are placed in direct contact with ocular bodily tissues and can tear or abrade such tissues, irritating or causing other tissue trauma. It has been found that the irritation caused by certain therapeutic agents or structure irregularities can be minimized by the use of one or more comforting agents. Furthermore, some patients will not tolerate punctum plugs as well as others. The initial discomfort of a punctum plug can be mitigated by the comforting agents, thus increasing retention time (otherwise diminished due to, for example, patient rubbing). Finally, in treatment of dry eye, the comforting agents can directly minimize the symptoms of the condition. The comforting agents are released into the tear film from the punctum plug.

Comforting agents include demulcents and emollients. A demulcent is an agent, usually a water-soluble polymer, which can be applied topically to the eye to protect and lubricate mucous membrane surfaces and relieve dryness and irritation. An emollient is an agent, usually a fat or oil, which can be applied locally to the eye to protect or soften tissues and to prevent drying and cracking.

Demulcents useful in the punctum plugs described herein include, but are not limited to: cellulose derivatives, dextran, gelatin, polyols, glycerin, polethylene glycol, polysotbate, propylene glycol, polyvinyl alcohol, and povidone, in particular opthalmic demulcents that are listed in 21 CFR §349.12, herein incorporated by reference. For example, demulcents that can be used in the punctum plugs described herein include carboxymethylcellulose sodium; dextrans (including dextran 70); gelatin; glycerin; hydroxyethyl cellulose; hydroxypropyl methylcellulose (HPMC); methylcellulose; polyethylene oxide; polyethylene glycol 300; polyethylene glycol 400; polysorbate 80; polyvinyl alcohol (PVA); povidone (polyvinylpyrrolidone); and propylene glycol.

Emollients useful in the punctum plugs described herein include, but are not limited to: lanolin preparation and oleaginous ingredients, in particular opthalmic emollients that are listed in 21 CFR §349.14, herein incorporated by reference. For example, ophthalmic emollients that can be used in the punctum plugs as described herein include: anhydrous lanolin; lanolin; light mineral oil; mineral oil; paraffin; petrolatum (up to 100 percent); white ointment; white petrolatum; white wax; and yellow wax.

Comforting agents also include lubricating compounds, such as simethicone. Comforting agents can also include sodium hyaluronate.

The comforting agent can be present in the plug body, coated on the surface of the plug, or both. In some embodiments, the comforting agent is embedded in the polymer of the plug body. In some embodiments, the comforting agent is mixed throughout the polymer of the plug body. The comforting agent can be mixed directly into the plug material by methods such as mechanical mixing, shearing, melt mixing, or ultrasound mixing. In some embodiments, the comforting agent is infused into the plug body during the molding process. The comforting agent can be chemically incorporated into the plug material by impregnating the plug using solvents or chemically bonding the comforting agent. In punctum plugs having a core structure, the comforting agent can be present in the core, the plug body, or both. In some embodiments, the comforting agent is mixed with a therapeutic agent in the plug body or core. In certain embodiments, more than one comforting agent and/or more than one therapeutic agent are released from the plug body, core, or both.

The release of comforting agent from the punctum plugs described herein can be controlled by the specific dimensions of the plug, the makeup of the plug, the concentration of comforting agent, molecular mass of the comforting agent, the hydrophylicity of the comforting agent, the properties of the supporting matrix whether the comforting agent is present in the plug body only, the optional core only, or both. Additionally, the amount and location of the optional therapeutic agent can affect the rate of release of the comforting agent from the plug. The rate of release of the therapeutic agent and the comforting agent from the punctual plug can be the same or different.

The comforting agent can be released from the punctum plug for approximately one day, approximately two days, approximately three days, approximately four days, approximately five days, approximately six days, approximately seven days, approximately eight days, approximately nine days, approximately ten days, approximately eleven days, approximately twelve days, approximately thirteen days, approximately fourteen days, approximately fifteen days, approximately sixteen days, approximately seventeen days, approximately eighteen days, approximately nineteen days, approximately twenty days, approximately twenty-one days, approximately twenty-two days, approximately twenty-three days, approximately twenty-four days, approximately twenty-five days, approximately twenty-six days, approximately twenty-seven days, approximately twenty-eight days, approximately twenty-nine days, or approximately thirty days, after insertion of the punctum plug.

### Sheath Body:

The sheath body can comprise appropriate shapes and materials to control the migration of therapeutic and/or comforting agent from the optional core. In some embodiments, the sheath body houses the core and can fit snugly against the core. The sheath body is made from a material that is substantially impermeable to the therapeutic and/or comforting agent so that the rate of migration of agent may be largely controlled by the exposed surface area of the core that is not covered by the sheath body. In many embodiments, migration of the agent through the sheath body can be about one tenth of the migration of agent through the exposed surface of the core, or less, often being one hundredth or less. In other words, the migration of the agent through the sheath body is at least about an order of magnitude less that the migration of agent through the exposed surface of the core. Suitable sheath body materials include polyimide, polyethylene terephthalate (hereinafter “PET”), polycarbonate, and polymethylmethacrylate (PMMA). The sheath body has a thickness, as defined from the sheath surface adjacent the core to the opposing sheath surface away from the core, from about 0.00025″ to about 0.0015″. The total diameter of the sheath that extends across the core ranges from about 0.2 mm to about 1.2 mm. The core may be formed by dip coating the core in the sheath material. Alternatively or in combination, the sheath body can comprise a tube and the core introduced into the sheath, for example as a liquid or solid that can be slid, injected or extruded into the sheath body tube. The sheath body can also be dip coated around the core, for example dip coated around a pre-formed core.

The sheath body can be provided with additional features to facilitate clinical use of the implant. For example, the sheath may receive a core that is exchangeable while the implant body, retention structure and sheath body remain implanted in the patient. The sheath body is often rigidly attached to the retention structure as described above, and the core is exchangeable while the retention structure retains the sheath body. In specific embodiments, the sheath body can be provided with external protrusions that apply force to the sheath body when squeezed and eject the core from the sheath body. Another core can then be positioned in the sheath body. In many embodiments, the sheath body or retention structure may have a distinguishing feature, for example a distinguishing color, to show placement such that the placement of the sheath body or retention structure in the canaliculus or other body tissue structure can be readily detected by the patient. The retention element or sheath body may comprise at least one mark to indicate the depth of placement in the canaliculus such that the retention element or sheath body can be positioned to a desired depth in the canaliculus based on the at least one mark.

### Retention Structure:

In many embodiments, a retention structure is employed to retain the implant in the punctum or canaliculus. The retention structure is attached to or integral with the implant body. The retention structure comprises an appropriate material that is sized and shaped so that the implant can be easily positioned in the desired tissue location, for example, the punctum or canaliculus. In some embodiments, the core may be attached to the retention structure via, at least in part, the sheath. In some embodiments, the retention structure comprises a hydrogel configured to expand when the retention structure is placed in the punctum. The retention structure can comprise an attachment member having an axially oriented surface. In some embodiments, expansion of the hydrogel can urge against the axially oriented surface to retain the hydrogel while the hydrogel is hydrated. In some embodiments, the attachment member can comprise at least one of a protrusion, a flange, a rim, or an opening through a portion of the retention structure. In some embodiments, the retention structure includes an implant body portion size and shape to substantially match an anatomy of the punctum and canaliculus.

The retention structure may have a size suitable to fit at least partially within the canalicular lumen. The retention structure can be expandable between a small profile configuration suitable for insertion and a large profile configuration to anchor the retention structure in the lumen, and the retention structure can be attached near the distal end of the core. In specific embodiments, the retention structure can slide along the core near the proximal end when the retention structure expands from the small profile configuration to the large profile configuration. A length of the retention structure along the core can be shorter in the large profile configuration than the small profile configuration.

In some embodiments, the retention structure is resiliently expandable. The small profile may have a cross section of no more than about 0.2 mm, and the large profile may have a cross section of no more than about 2.0 mm. The retention structure may comprise a tubular body having arms separated by slots. The retention structure can be disposed at least partially over the core.

In some embodiments, the retention structure is mechanically deployable and typically expands to a desired cross sectional shape, for example with the retention structure comprising a super elastic shape memory alloy such as Nitinol™. Other materials in addition to Nitinol™ can be used, for example resilient metals or polymers, plastically deformable metals or polymers, shape memory polymers, and the like, to provide the desired expansion. In some embodiments polymers and coated fibers available from Biogeneral, Inc. of San Diego, Calif. may be used. Many metals such as stainless steels and non-shape memory alloys can be used and provide the desired expansion. This expansion capability permits the implant to fit in hollow tissue structures of varying sizes, for example canaliculae ranging from 0.3 mm to 1.2 mm (i.e. one size fits all). Although a single retention structure can be made to fit canaliculae from 0.3 to 1.2 mm across, a plurality of alternatively selectable retention structures can be used to fit this range if desired, for example a first retention structure for canaliculae from 0.3 to about 0.9 mm and a second retention structure for canaliculae from about 0.9 to 1.2 mm. The retention structure has a length appropriate to the anatomical structure to which the retention structure attaches, for example a length of about 3 mm for a retention structure positioned near the punctum of the canaliculus. For different anatomical structures, the length can be appropriate to provide adequate retention force, e.g. 1 mm to 15 mm lengths as appropriate.

Although the implant body may be attached to one end of the retention structure as described above, in some embodiments the other end of the retention structure is not attached to the implant body so that the retention structure can slide over the implant body including the sheath body and core while the retention structure expands. This sliding capability on one end can be desirable as the retention structure may shrink in length as the retention structure expands in width to assume the desired cross sectional width. However, it should be noted that some embodiments may employ a sheath body that does not slide in relative to the core.

In various embodiments, the retention structure can be retrieved from tissue. A projection, for example a hook, a loop, or a ring, can extend from a portion of the implant body to facilitate removal of the retention structure.

In some embodiments, the sheath and retention structure can comprise two parts.

### Occlusive Element:

An occlusive element can be mounted to and expandable with the retention structure to inhibit tear flow. An occlusive element may inhibit tear flow through the lumen, and the occlusive element may cover at least a portion of the retention structure to protect the lumen from the retention structure. The occlusive element comprises an appropriate material that is sized and shaped so that the implant can at least partially inhibit, even block, the flow of fluid through the hollow tissue structure, for example lacrimal fluid through the canaliculus. The occlusive material may be a thin walled membrane of a biocompatible material, for example silicone, that can expand and contract with the retention structure. The occlusive element is formed as a separate thin tube of material that is slid over the end of the retention structure and anchored to one end of the retention structure as described above. Alternatively, the occlusive element can be formed by dip coating the retention structure in a biocompatible polymer, for example silicone polymer. The thickness of the occlusive element can be in a range from about 0.01 mm to about 0.15 mm, and often from about 0.05 mm to 0.1 mm.

Comforting Agent and/or Therapeutic Agent Core:

The optional core may be inserted into an implant body, or may serve as the implant itself, without any additional structural components. The core can include a therapeutic agent or multiple therapeutic agents and/or the comforting agent. The core comprises materials to provide release of the therapeutic and/or comforting agent. In some embodiments, the core comprises a sustained release formulation, which formulation consists of or consists essentially of comforting agent and/or therapeutic agent and silicone as a carrier. The agent migrates from the core to the target tissue, for example ciliary muscles of the eye. The core may optionally comprise an agent or agents in a matrix, wherein the agent is dispersed or dissolved within the matrix. The agent or agents may be only slightly soluble in the matrix so that a small amount is dissolved in the matrix and available for release from the surface of the core. As the agent diffuses from the exposed surface of the core to the tear or tear film, the rate of migration from the core to the tear or tear film can be related to the concentration of agent dissolved in the matrix. In addition or in combination, the rate of migration of agent from the core to the tear or tear film can be related to properties of the matrix in which the agent is dissolved.

In an embodiment, the topical formulation or the core does not contain a preservative. Preservatives include, for example, benzalkonium chloride and EDTA. In an embodiment, the implants may be less allergenic and may reduce chemical sensitivity compared to formulations containing these preservatives.

In specific embodiments, the rate of migration from the core to the tear or tear film can be based on a silicone formulation. In some embodiments, the concentration of comforting and/or therapeutic agent dissolved in the core may be controlled to provide the desired rate of release of the agent. The agent included in the core can include liquid (such as oil), solid, solid gel, solid crystalline, solid amorphous, solid particulate, or dissolved forms of the agent. In a some embodiments, the core may comprise liquid or solid inclusions, for example liquid Latanoprost droplets dispersed in the silicone matrix.

Table 1 shows core silicones that may be used and associated cure properties, according to embodiments of the present subject matter. The core insert matrix material can include a base polymer comprising dimethyl siloxane, such as MED-4011, MED 6385 and MED 6380, each of which is commercially available from NuSil. The base polymer can be cured with a cure system such as a platinum-vinyl hydride cure system or a tin-alkoxy cure system, both commercially available from NuSil. In many embodiments, the cure system may comprise a known cure system commercially available for a known material, for example a known platinum vinyl hydride cure system with known MED-4011. In a specific embodiment shown in Table 1, 90 parts of MED-4011 can be combined with 10 parts of the crosslinker, such that the crosslinker comprises 10% of the mixture. A mixture with MED-6385 may comprise 2.5% of the crosslinker, and mixtures of MED-6380 may comprise 2.5% or 5% of the crosslinker.

TABLE 1
Core Silicone Selections

| | | | Crosslinker |
|---|---|---|---|
| Material | Base Polymer | Cure System | Percent |
| MED-4011 | Dimethyl siloxane | Platinum vinyl | 10% |
| | Silica filler | hydride system | |
| | material | 10% | |
| MED-6385 | Dimethyl siloxane | Tin-Alkoxy 2.5% | 2.5% |
| | Diatomaceous | | |
| | earth filler material | | |
| MED-6380 | Dimethyl siloxane | Tin-Alkoxy | 2.5 to 5% |
| | without filler | | |
| | material | | |

It has been found that the cure system and type of silicone material can affect the curing properties of the solid core insert, and may potentially affect the yield of therapeutic and/or comforting agent from the core matrix material. In specific embodiments, curing of MED-4011 with the platinum vinyl hydride system can be inhibited with high concentrations of therapeutic agent drug/prodrug and/or comforting agent, for example over 20% agent, such that a solid core may not be formed. In specific embodiments, curing of MED-6385 or MED 6380 with the tin alkoxy system can be slightly inhibited with high concentrations, e.g. 20%, of agent. This slight inhibition of curing can be compensated by increasing the time or temperature of the curing process. For example, embodiments of the present subject matter can make cores comprising 40% agent and 60% MED-6385 with the tin alkoxy system using appropriate cure times and temperatures. Similar results can be obtained with the MED-6380 system the tin-alkoxy system and an appropriate curing time or temperature. Even with the excellent results for the tin alkoxy cure system, it has been found that there may be an upper limit, for example 50% agent or more, at which the tin-alkoxy cure system may not produce a solid core. In many embodiments, the therapeutic and/or comforting agent in the solid core may be at least about 5%, for example a range from about 5% to 50%, and can be from about 20% to about 40% by weight of the core.

The core or other agent supply (e.g., implant impregnated body) can comprise one or more biocompatible materials capable of providing release of agent. Although the core is described above with respect to an embodiment comprising a matrix with a substantially non-biodegradable silicone matrix with inclusions of agent located therein that dissolve, the core can include structures that provide sustained release of agent, for example a biodegradable matrix, a porous core, liquid cores and solid cores.

A matrix that contains therapeutic and/or comforting agents can be formed from either biodegradable or non-biodegradable polymers. A non-biodegradable core can include silicone, acrylates, polyethylenes, polyurethane, polyurethane, hydrogel, polyester (e.g., DACRON® from E. I. Du Pont de Nemours and Company, Wilmington, Del.), polypropylene, polytetrafluoroethylene (PTFE), expanded PTFE (ePTFE), polyether ether ketone (PEEK), nylon, extruded collagen, polymer foam, silicone rubber, polyethylene terephthalate, ultra high molecular weight polyethylene, polycarbonate urethane, polyurethane, polyimides, stainless steel, nickel-titanium alloy (e.g., Nitinol), titanium, stainless steel, cobalt-chrome alloy (e.g., ELGILOY® from Elgin Specialty Metals, Elgin, Ill.; CONICHROME® from Carpenter Metals Corp., Wyomissing, Pa.).

A biodegradable core can comprise one or more biodegradable polymers, such as protein, hydrogel, polyglycolic acid (PGA), polylactic acid (PLA), poly(L-lactic acid) (PLLA), poly(L-glycolic acid) (PLGA), polyglycolide, poly-L-lactide, poly-D-lactide, poly(amino acids), polydioxanone, polycaprolactone, polygluconate, polylactic acid-polyethylene oxide copolymers, modified cellulose, collagen, polyorthoesters, polyhydroxybutyrate, polyanhydride, polyphosphoester, poly(alpha-hydroxy acid) and combinations thereof. In some embodiments the core can comprise at least one hydrogel polymer. In some embodiments, the core includes a thin-walled polyimide tube sheath body that is filled with a therapeutic and/or comforting agent dispersed in Nusil 6385, a cured medical grade solid silicone. The cured silicone serves as the solid, non-erodible matrix from which agent slowly elutes. The core is sealed at the distal end with a cured film of solid Loctite 4305 medical grade adhesive (cyanoacrylate). The polyimide tube sheath body is inert and, together with the adhesive, provides structural support and a barrier to both lateral agent diffusion and agent diffusion through the distal end of the insert. The insert is seated in the bore of the punctum plug and is held in place via an interference fit.

### Specific Implant Embodiments

Various embodiments of the implant that may be employed in the methods described herein are as follows.

In various embodiments, the plug can be a “half and half”, or a “stuff” plug design, wherein a hydrogel-forming polymer disposed as indicated forms a junction with a substantially non-swelling polymer. In the stuff plug design, the substantially non-swelling polymer surrounds a core of hydrogel-forming material. In the half and half design, the hydrogel-forming material is uncovered. In both designs, an adhesive junction between the first polymer and the second polymer, optionally comprising a third polymer which can exhibit an intermediate degree of swelling in an aqueous medium, is sufficiently strong to hold the plug intact under a degree of tension, such as when the plug is removed from the punctal canal.

The plug can include a cylindrical design wherein approximately one half, the distal half when disposed in the punctal canal, comprises the hydrogel-forming polymer, and the other half, the proximal half when disposed in the punctal canal, comprises the non-swelling (or minimally-swelling) polymer that can contain a therapeutic agent, a comforting agent, or both. The proximal half of the plug can be adapted to receive a core containing a therapeutic agent, a comforting agent, or both. In some embodiments, the junction between the first and second plug portions can include joined butt ends of the two cylindrical segments.

To increase the surface area of the junction between the first portion and the second portion, the plug can be adapted such that a greater relative surface of contact is present between the two polymer materials. For example, a “skeleton” design can be used wherein a segment of the first portion extends within a sheath of the hydrogel-forming material. This segment can be a rod or mandrel, wherein the hydrogel-forming polymer is disposed around the rod, and the surface area of contact is the surface area of the exterior of the rod segment. Alternatively, the segment can be further modified to even more greatly increase the surface area of contact, such as by the presence of discs of the non-swelling polymer, around which a body of the swelling polymer is disposed. Alternatively, the modifications of the mandrel can include “ribs,” “spokes,” or “fishhooks,” all serving the purpose of increasing the surface area of contact between the rod or mandrel and the hydrogel sheath.

The first and second polymers of the plug portions can be bonded at the increased surface area junction between the first and second portions of the plug. Optionally, a third polymer exhibiting an intermediate degree of swelling in an aqueous medium can be disposed on the entire junction surface, or on a section of the junction surface. In this way, the junction can be made sufficiently strong to avoid separation of the two portions when the plug is removed from the punctal canal by a physician. Alternatively, a non-swelling third polymer can have a hole that can be embedded in the hydrogel-forming second polymer such that a continuous volume of the second polymer extends through the hole and further strengthens the bond between the first and second portions of the plug. Or, the mandrel with the hole could be made of a fourth material that has a higher durometer (i.e., is harder) than the other materials already mentioned. This material can be a urethane-carbonate co-polymer, or polycarbonate, or other composite materials. The harder material provides increased plug integrity and mechanical strength.

Upon insertion of the plug into the punctal canal, the second portion of the plug can come in contact with the tear fluid that normally drains through the punctal canal. Upon contact, the second polymer, a hydrogel-forming material, absorbs water and swells to help hold the plug within the canal. The first portion, comprising the non-swelling or minimally-swelling first polymer, remains bonded to the hydrogel material while the plug is held within the punctal canal.

To further increase adhesion between the two polymers, the junction surface can be chemically treated, for example by oxidation, such that the bond between the two portions of the plug is strengthened.

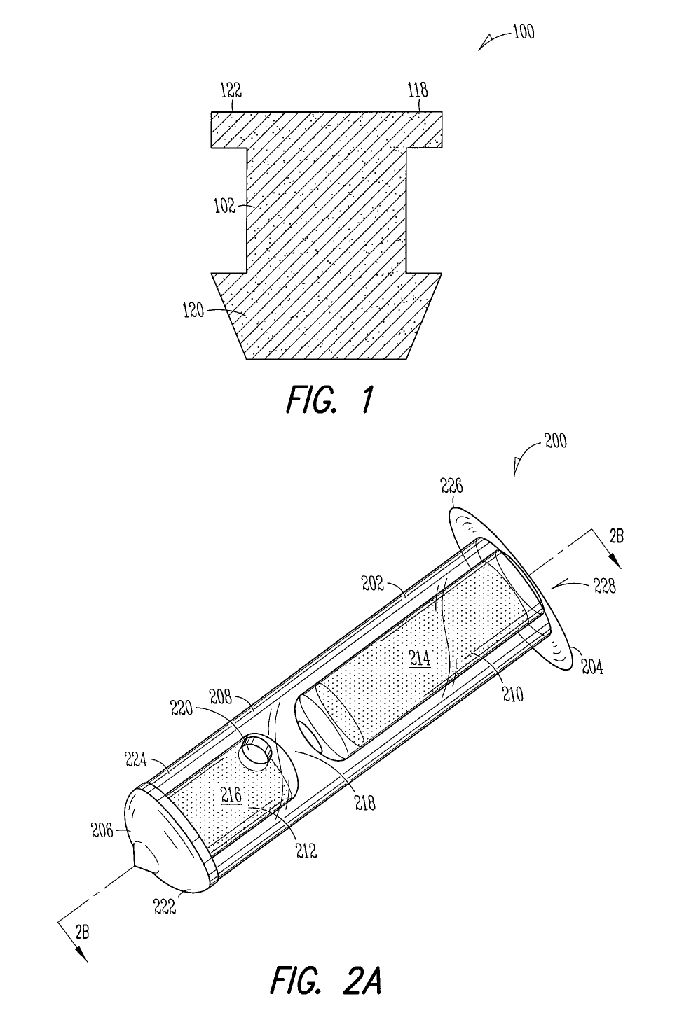

FIG. 1 illustrates an example embodiment of a cross-sectional view of a punctum plug 100 taken along a line parallel to a longitudinal axis of the plug. As shown in FIG. 1, the punctum plug 100 comprises a plug body 102. In the embodiment shown, the plug body 102 includes an integral feedback or other projection 122, such as a projection extending laterally at least partially from or around a proximal end 118 of the plug body 102. The projection 122 is in the form of a collarette extending radially outwardly from the plug body 102 to a degree sufficient so that at least a portion of the collarette will extend beyond and be exterior to the punctum after insertion of plug body 102 distal portions into the canaliculus.

In this embodiment, the plug body 102 is at least partially impregnated with a comforting agent-releasing or therapeutic agent-releasing supply 120. In certain embodiments, the agent supply 120 is disposed within, dispersed throughout, or otherwise contained in the plug body 102. As discussed in commonly-owned Odrich, application Ser. No. 10/825,047 (filed Apr. 15, 200 and entitled Drug Delivery via Punctal Plug), which is herein incorporated by reference in its entirety, the agent of the supply 120 can be released from the plug body 102 into tear fluid of the eye or into the nasolacrimal duct system. In some embodiments, an impermeable sheath is disposed over portions of the plug body 102 to control agent supply 120 release therefrom.

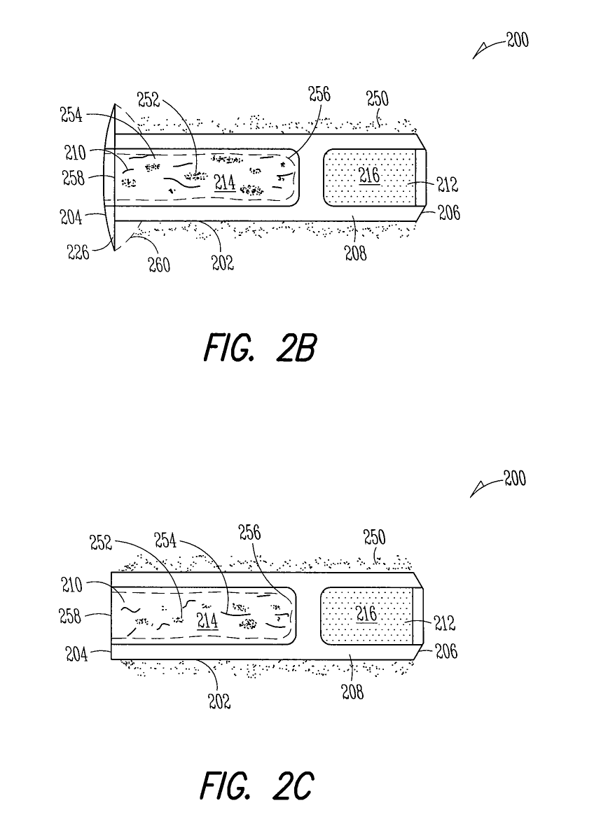

FIG. 2A illustrates an example embodiment of a punctum plug implant 200 that is insertable into a lacrimal punctum. The insertion of the punctum plug implant 200 into the lacrimal punctum allows for one or more of inhibition or blockage of tear flow therethrough (e.g., to treat dry eyes) or the delivery of an agent or agents to an eye (e.g., to treat one or more of infection, inflammation, glaucoma or other ocular diseases). In this embodiment, the punctum plug 200 comprises a plug body 202 extending from a proximal end portion 204 to a distal end portion 206 and having a retention structure 208.

In various embodiments, the plug body 202 can comprise an elastic material, such as silicone, polyurethane or other urethane-based material, or an acrylic of a non-biodegradable, partially biodegradable or biodegradable nature (i.e., erodeable within the body) allowing at least one portion of the retention structure to deform outward. In some embodiments, the biodegradable elastic materials include cross-linked polymers, such as poly (vinyl alcohol). In some embodiments, different portions of the plug body 202 are made of different materials. For instance, the plug body proximal end portion 204 can comprise a silicone/polyurethane co-polymer and the plug body distal end portion 206 can comprise a polyurethane hydrogel or other solid hydrogel. In certain embodiments, the plug body proximal end portion 204 can comprise silicone and the plug body distal end portion 206 can comprise a hydrophilic silicone mixture. Other co-polymers that can be used to form the plug body 302 include silicone/urethane, silicone/poly(ethylene glycol) (PEG), and silicone/2hydroxyethyl methacrylate (HEMA).

In certain embodiments, the plug body 202 can include a cylindrical-like structure having a first chamber 210 at or near the proximal end and a second chamber 212 at or near the distal end. A core 214 can be disposed in the first chamber 210, while a hydrogel or other expandable retention element 216 of a biodegradable or non-biodegradable nature can be disposed in the second chamber 216. In some embodiments, the biodegradable retention elements include salt and cellulose based mixtures. In some embodiments, the non-biodegradable retention elements include hydrogels or other synthetic polymers. A plug body septum 218 can be positioned between the first chamber 210 and the second chamber 216 and can be used to inhibit or prevent communication of a material between the core 214 and the hydrogel retention element 216.

In various ways, the expandable, hydrogel retention element 216 can be substantially encapsulated, such as within a portion of the retention structure 208. In various embodiments, the retention structure 208 can include a fluid permeable retainer allowing fluid to be received into and absorbed or otherwise retained by the hydrogel retention element 216, such as upon its insertion into the punctum. The hydrogel retention element 216 can be configured to expand, such as to a size or shape that urges one or more outer surface portions of the retention structure 208 to contact a wall of the lacrimal canaliculus, thereby retaining or helping retain a least a portion of the plug implant within the punctum. In some embodiments, the fluid permeable retainer can include a fluid permeable aperture 220, such as disposed in a lateral wall of the retention structure 208. In some embodiments, the fluid permeable retainer can include a fluid permeable or hydrophilic cap member 222 or other membrane. In some embodiments, the fluid permeable retainer can include a fluid permeable or hydrophilic plug body portion 224. These examples of fluid permeable retainers 220, 222, and 224 can also inhibit the hydrogel retention element 216 from appreciably protruding out of the retention structure 208 during and upon expansion.

The plug implant body 202 can include a feedback or other projection 226, such as extending laterally at least partially from or around (e.g., a removal loop) a proximal end portion 204 of the plug body 202. In some embodiments, the projection 226 can include a removal loop. In some embodiments, the projection 226 can be configured to seat against or near (e.g., via a ramped portion 260 (FIG. 2B)) the punctum opening, such as for inhibiting or preventing the punctum plug 200 from passing completely within the canaliculus, or for providing tactile or visual feedback information to an implanting user regarding the same. In some embodiments, a proximal end of the projection 226 can include a convex such as for helping provide comfort to a patient when implanted. In some embodiments, the projection 226 can include a convex radius of about 0.8 millimeters. In some embodiments, the projection 226 is between about 0.7 millimeters to about 0.9 millimeters in diameter. In some embodiments, the projection 226 can include a non-concave shape of about 0.5 millimeters to about 1.5 millimeters in diameter, and 0.1 millimeters to about 0.75 millimeters in thickness. In some embodiments, the projection 226 has a wing-like shape, in which a column-like projection extends from opposite sides of the implant plug proximal end 204. In some examples, the projection 226 includes a partially trimmed collar extending 360 degrees around the proximal end 204 from an outer plug body surface. In some examples, such the projection 226 includes a full collar extending 360 degrees around the proximal end 204 from an outer plug body surface. In an example, the projection 226 includes a cross-sectional shape similar to a flat disk (i.e., relatively flat top and bottom surfaces). A drug or other agent elution port 228 can extend though the projection 226, such as to provide sustained release of a core 214 agent or agents onto an eye.

FIG. 2B illustrates a cross-sectional view of an example embodiment of a punctum plug implant 200 taken along a line parallel to a longitudinal axis of the implant, such as along line 2B-2B of FIG. 2A. As shown in FIG. 2B, the punctum plug can include a plug body 202 having a retention structure 208 substantially encapsulating a hydrogel retention element 216 at or near a plug body distal end portion 206, and a therapeutic and/or comforting agent core 214 disposed within the plug body, for example at or near a proximal end portion 204. In this embodiment, the core 214 is disposed in a first plug body chamber 210 and the hydrogel retention element 216 is disposed in a second plug body chamber 212. As discussed above, the hydrogel retention element 216 can be configured to expand to a size or shape that retains or helps retain at least a portion of the plug implant 200 within the lacrimal punctum. In some embodiments, a hydrogel retention element 250 can also be coated or otherwise provided on an outer surface portion of the plug body 202 providing another mechanism for retaining or helping to retain at least a portion of the plug 200 at least partially within the lacrimal punctum.

The retention structure 208, which can be used to substantially encapsulate the hydrogel retention element 216, can be of varying sizes relative to a plug body 202 size. In some embodiments, the retention structure 208 is at least about one fifth the length of the plug body 202. In some embodiments, the retention structure 208 is at least about one fourth the length of the plug body 202. In some embodiments, the retention structure 208 is at least about one third the length of the plug body 202. In some embodiments, the retention structure 208 is at least about one half the length of the plug body 202. In some embodiments, the retention structure 208 is at least about three quarters the length of the plug body 202. In some embodiments, the retention structure 208 is about the full length of the plug body 202.

As shown in the example embodiment of FIG. 2B, the hydrogel retention element 216 can have a non-expanded, “dry” state, which aids insertion through the punctum and into the lacrimal canaliculus. Once placed in the canaliculus, the hydrogel retention element 216 can absorb or otherwise retain canalicular or other fluid, such as via a fluid permeable retainer 220, 222, 224 (FIG. 2A) to form an expanded structure. In some embodiments, the hydrogel retention element 216 can include a material that is non-biodegradable. In some embodiments, the hydrogel retention element 216 can include a material that is biodegradable. Other options for the hydrogel retention element 216 can also be used. For instance, the hydrogel retention element 216 can be molded with the retention structure 208 in a single piece, or can be formed separately as one piece and subsequently coupled to the retention structure 208.

In some embodiments, the therapeutic and/or comforting agent core 214 disposed at or near the proximal end portion 204 of the plug body 202 can include a plurality of agent inclusions 252, which can be distributed in a matrix 254. In some embodiments, the inclusions 252 comprise a concentrated form of the agent (e.g., a crystalline agent form). In some embodiments, the matrix 254 can comprise a silicone matrix or the like, and the distribution of inclusions 252 within the matrix can be non-homogeneous. In some embodiments, the agent inclusions 252 include droplets of an oil, such as latanoprost oil. In still other embodiments, the agent inclusions 252 comprise solid particles. The inclusions can be of many sizes and shapes. For instance, the inclusions can be microparticles having dimensions on the order of about 1 micrometers to about 100 micrometers.

In the embodiment shown, the core 214 has a sheath body 256 disposed over at least a portion thereof such as to define at least one exposed surface 258 of the core. The exposed surface 258 can be located at or near the proximal end portion 204 of the plug body such as to contact a tear or a tear film fluid and release the comforting and/or therapeutic agent at one or more therapeutic levels over a time period when the punctum plug 200 is inserted into the punctum.

FIG. 2C illustrates a cross-sectional view of an example embodiment of a punctum plug 200 taken along a line parallel to a longitudinal axis of the plug. As shown in FIG. 2C, the punctum plug includes a plug body 202 without a feedback or other projection 226 (FIG. 2A). In this way, the plug 200 can be completely inserted inside the lacrimal punctum. In some embodiments, the first chamber 210 can include dimensions of about 0.013 inches×about 0.045 inches. In some embodiments, the second chamber 212 can include dimensions of about 0.013 inches by about 0.020 inches.

FIG. 3A illustrates another embodiment of a punctum plug implant 300 that can be insertable into a lacrimal punctum. The insertion of the punctum plug 300 into the lacrimal punctum can allow for one or more of: inhibition or blockage of tear flow therethrough (e.g., to treat dry eyes) or the delivery of a therapeutic and/or comforting agent to an eye (e.g., to treat an infection, irritation, inflammation, glaucoma or other ocular disease or disorder), a nasal passage (e.g., to treat a sinus or allergy disorder) or an inner ear system (e.g., to treat dizziness or a migraine).

In this embodiment, the punctum plug 300 comprises a plug body 302 including first 304 and second 306 portions. The plug body 302 extends from a proximal end 308 of the first portion 304 to a distal end 310 of the second portion 306. In various embodiments, the proximal end 308 can define a longitudinal proximal axis 312 and the distal end 310 can define a longitudinal distal axis 314. The plug body 300 can be configured such that, when implanted, an at least 45 degree angled intersection 316 exists between the proximal axis 312 and the distal axis 314 for biasing at least a portion of the plug body 302 against at least a portion of a lacrimal canaliculus located at or more distal to a canaliculus curvature. In some embodiments, the plug body 302 can be configured such that the angled intersection 316 is between about 45 degrees and about 135 degrees. In this embodiment, the plug body 302 is configured such that the angled intersection 316 is approximately about 90 degrees. In various embodiments, a distal end 326 of the first portion 304 can be integral with the second portion 306 at or near a proximal end 328 of the second portion 306.

In certain embodiments, the plug body 302 can include angularly disposed cylindrical-like structures comprising one or both of a first cavity 318 disposed near the proximal end 308 or a second cavity 320 disposed near the distal end 310. In this embodiment, the first cavity 318 extends inward from the proximal end 308 of the first portion 304, and the second cavity 320 extends inward from the distal end 310 of the second portion 306. A first agent supply 322 can be disposed in the first cavity 318 to provide agent release to an eye, while a second drug-releasing or other agent-releasing drug supply 324 can be disposed in the second cavity 320 to provide a drug or other agent release to a nasal passage or inner ear system, for example. A plug body septum 330 can be positioned between the first cavity 318 and the second cavity 320, and can be used to inhibit or prevent communication of a material between the first agent supply 322 and the second agent supply 324.

In some embodiments, the therapeutic and/or comforting agent release can occur, at least in part, via an exposed surface of the supply 322, 324. Description regarding effective release rates of one or more drugs or other agents from a supply 322, 324 can be found in commonly-owned DeJuan et al., U.S. application Ser. No. 11/695,545 (filed Apr. 2, 2007 and entitled Nasolacrimal Drainage System Implants for Drug Therapy) which is herein incorporated by reference in its entirety, including its description of obtaining particular release rates. In some embodiments, the exposed surface of the supply 322, 324 can be flush or slightly below the proximal end 308 of the first portion 304 or the distal end 310 of the second portion 306, respectively, such that the agent supply does not protrude outside of the plug body 302. In some embodiments, the exposed surface of the agent supply 322, for instance, can be positioned above the proximal end 308 such that the agent supply 322 at least partially protrudes outside of the plug body 302.

The plug body 302 can include an integral feedback or other projection 332, such as projections extending laterally at least partially from or around a proximal end 308 of the first plug body portion 304. In some embodiments, the projection 332 can include a set of wings for use in removing the punctum plug 300 from an implant position. The removal set of wings can be configured without migration in mind, as the non-linear configuration of the plug body 302 can prevent migration by assuming a size or shape of the canaliculus curvature and optionally, the lacrimal canaliculus ampulla. In some embodiments, the projection 332 can be configured to seat against or near the punctal opening such as for inhibiting or preventing the punctum plug 300 from passing completely within the lacrimal canaliculus, or for providing tactile or visual feedback information to an implanting user, e.g., as to whether the plug is fully implanted. The projection 332 can extend laterally in a direction parallel to or away from an eye when implanted. This will reduce irritation to the eye as compared to a case in which a portion of the projection extends toward the eye. In addition, a lateral extension direction of the projection 332 from the proximal end 308 can be substantially the same as a lateral extension direction of the second plug body portion 306 relative to the distal end 326 of the first plug body portion 304. This can also avoid extension toward the eye. A drug or other agent elution port can extend though a collar-projection 332, such as to provide sustained release of the supply 322 agent onto an eye.

In various embodiments, the plug body 302 can be molded using an elastic material, such as silicone, polyurethane, NuSil (e.g., NuSil 4840 with 2% 6-4800) or an acrylic of a non-biodegradable, partially biodegradable or biodegradable nature (i.e., erodeable within the body) allowing a non-linear extending plug body 302 to be formed. In some embodiments, the biodegradable elastic materials can include cross-linked polymers, such as poly (vinyl alcohol). In some embodiments, the plug body 302 can comprise a silicone/polyurethane co-polymer. Other co-polymers that can be used to form the plug body 302 include, but are not limited to, silicone/urethane, silicone/poly (ethylene glycol) (PEG), and silicone/2hydroxyethyl methacrylate (HEMA). As discussed in commonly-owned Jain et al., Application Ser. No. 61/049,317 (filed Apr. 30, 2008 and entitled Drug-Releasing Polyurethane Lacrimal Insert), which is herein incorporated by reference in its entirety, urethane-based polymer and copolymer materials allow for a variety of processing methods and bond well to one another.

FIG. 3B illustrates an example embodiment of a cross-sectional view of a punctum plug 300 taken along a line parallel to a longitudinal axis of the plug, such as along line 3B-3B of FIG. 3A. As shown in FIG. 3B, the punctum plug 300 can include a plug body 302 including first 304 and second 306 portions. The plug body 302 extends from a proximal end 308 of the first portion 304 to a distal end 310 of the second portion 306. In various embodiments, the proximal end 308 can define a longitudinal proximal axis 312 and the distal end 310 can define a longitudinal distal axis 314. The plug body 300 can be configured such that, when implanted, an at least 45 degree angled intersection 316 exists between the proximal axis 312 and the distal axis 314 for biasing at least a portion of the plug body 302 against at least a portion of a lacrimal canaliculus located at or more distal to a canaliculus curvature. In this embodiment, the plug body 300 is configured such that the angled intersection 316 is approximately about 90 degrees.

In various embodiments, a distal end 326 of the first portion 304 can be integral with the second portion 306 at or near a proximal end 328 of the second end 326. In some embodiments, the second portion 306 can include a length having a magnitude less than four times a length of the first portion 304. In one embodiment, the second portion 306 can include a length of less than about 10 millimeters, such as is shown in FIG. 3B. In another embodiment, the second portion 306 can include a length less than about 2 millimeters.

In certain embodiments, the second portion 306 can comprise an integral dilator 350 to dilate anatomical tissue 352, such as one or both of a lacrimal punctum or canaliculus to a sufficient diameter as the punctum plug 300 is being implanted. In this way, the punctum plug 300 can be implanted in various size ocular anatomies without the need for pre-dilation via a separate enlarging tool. The dilator 350 can be formed so as to not be traumatic to an inner lining of the punctum and the canaliculus. In some embodiments, a lubricious coating disposed on, or impregnated in, an outer surface of the plug body 302 can be used to further aid insertion of the punctum plug 300 into the anatomical tissue 352. In one embodiment, the lubricious coating can include a silicone lubricant.

As shown, the dilator 350 can generally narrow from a location near the proximal end 328 of the second portion 306 to the distal end 310 of the second portion 306, such as from a diameter of about 0.6 millimeters to a diameter of about 0.2 millimeters. In some embodiments, an outer surface slope of the dilator 350, as measured from the location near the proximal end 328 of the second portion 306 to the distal end 310 of the second portion 306, can be between about 1 degree and about 10 degrees (e.g., 2 degrees, 3 degrees, 4 degrees, or 5 degrees) with respect to the longitudinal distal axis 314. In some embodiments, the slope of the dilator 350 can be less than 45 degrees with respect to the longitudinal distal axis 314. Among other factors, a determination of a desirable dilator 350 slope for a given implant situation can be made by balancing a plug body 302 strength desirable for plug implant with a desire to have a soft, flexible and conforming plug body (e.g., to conform to a lacrimal canaliculus anatomy) upon implantation. In some embodiments, a diameter of a dilator tip 354 can be between about 0.2 millimeters and about 0.5 millimeters.

In certain embodiments, the proximal end 328 of the second plug body portion 306 can include a lead extension 356 configured to bias against at least a portion of a lacrimal canaliculus ampulla when implanted. In this embodiment, the lead extension 356 projects proximally from the intersection between the first 304 and second 306 plug body portions, such as in an opposite direction as the extension of the dilator 350.

In certain embodiments, the plug body 302 can include a first cavity 318 disposed near the proximal end 308. In this embodiment, the first cavity 318 extends inward about 2 millimeters or less from the proximal end 308, and houses a first therapeutic agent and/or comforting agent-releasing supply 322 to provide a drug or other agent release to an eye. In some embodiments, the supply 322 can include a plurality of therapeutic agent and/or comforting agent inclusions 360, which can be distributed in a matrix 362. In some embodiments, the inclusions 360 can comprise a concentrated form of the therapeutic agent (e.g., a crystalline agent form). In some embodiments, the matrix 362 can comprise a silicone matrix or the like, and the distribution of inclusions 360 within the matrix can be non-homogeneous. In some embodiments, the agent inclusions 360 can include droplets of oil, such as latanoprost oil. In still other embodiments, the agent inclusions 360 can comprise solid particles, such as Bimatoprost particles in crystalline form. The inclusions can be of many sizes and shapes. For instance, the inclusions can include microparticles having dimensions on the order of about 1 micrometer to about 100 micrometers.

In the embodiment shown, the agent supply 322 includes a sheath body 366 disposed over at least a portion thereof such as to define at least one exposed surface 368 of the agent supply. The exposed surface 368 can be located at or near the proximal end 308 of the plug body 302 such as to contact a tear or a tear film fluid and release the agent at one or more desired levels over a defined time period when the punctum plug 300 is inserted into the lacrimal punctum.

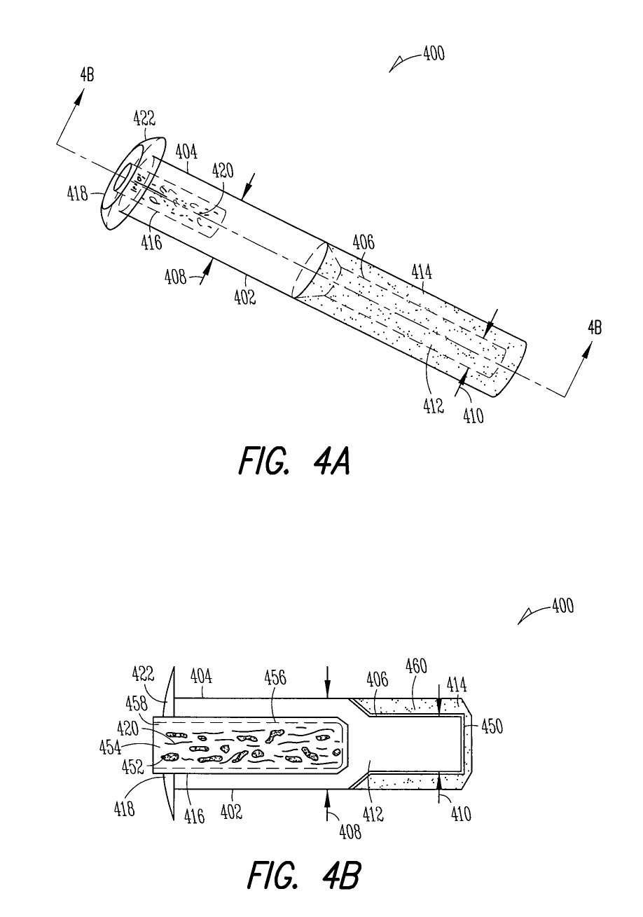

FIG. 4A illustrates an embodiment of a punctum plug 400 that can be insertable into a lacrimal punctum. In various embodiments, the punctum plug 400 comprises a plug body 402, including first 404 and second 406 portions, which is sized and shaped for at least partial insertion into a lacrimal punctum. The first portion 404 is formed from a polymer and includes a first diameter 408. The second portion 406 is also formed from a polymer and includes a base member 412 (e.g., mandrel or spine-like member) having a second diameter 410, which is less than the first diameter 408. In an embodiment, the first 404 and second 406 portions are integrally coupled and comprise a unitary plug body 402. In an embodiment, the first 404 and second 406 portions are separate elements, which can be coupled to one another via an engagement between a coupling void and a coupling arm, for instance.

An expandable retention member 414, such as a swellable material, can be bonded or otherwise coupled over the base member 412 such that it envelops, at least in part, a portion of the base member 412. In an embodiment, the expandable retention member substantially envelops the base member 412. As the expandable retention member 414 absorbs or otherwise retains lacrimal or other fluid, such as upon insertion into a lacrimal punctum, its size increases and its shape may change thereby urging itself against and slightly biasing a wall of the associated canaliculus. It is believed that the expandable retention member 414 will provide retention comfort to a subject and may improve punctum plug 400 implant retention via controlled biasing of the canaliculus wall.

The positioning of the expandable retention member 414 over a portion of the plug body 402 allows the retention member 414 to be freely exposed to lacrimal fluid in situ, thereby allowing for a wide range of potential expansion rates. Further, the base member 412 provides an adequate coupling surface area to which the expandable retention member 414, for example, can adhere such that the material of the expandable retention member 414 does not remain in a lacrimal punctum after the punctum plug 400 is removed from the subject. As shown in this embodiment, the expandable retention member 414 can include a non-expanded, “dry or dehydrated” state, which aids insertion through a lacrimal punctum and into the associated lacrimal canaliculus. Once placed into a lacrimal canaliculus, the expandable retention member 414 can absorb or otherwise retain lacrimal fluid to form an expanded structure.

In some embodiments, the plug body 402 can include a cylindrical-like structure comprising a cavity 416 disposed near a proximal end 418 of the first portion 404. In this embodiment, the cavity 416 extends inward from the proximal end 418 and includes a first therapeutic agent and/or comforting agent releasing supply 420 to provide release to an eye. The agent release can occur, at least in part, via an exposed surface of the supply 420. In an embodiment, the exposed surface of the agent supply 420 can be positioned above the proximal end 418 such that the agent supply 420 at least partially protrudes outside of the plug body 402. In some embodiments, the exposed surface of the agent supply 420 can be flush or slightly below the proximal end 418 such that the supply 420 does not protrude outside of the plug body 402.

The plug body 402 can include an integral feedback or other projection 422, such as projections extending laterally at least partially from or around the proximal end 418 of the first plug body portion 404. In an embodiment, the projection 422 includes a partially trimmed collar extending 360 degrees around the proximal end 418 from an outer plug body surface. In an embodiment, the projection 422 includes a full collar extending 360 degrees around the proximal end 418 from an outer plug body surface. In an embodiment, the projection 422 includes a cross-sectional shape similar to a flat disk (i.e., relatively flat top and bottom surfaces). In various embodiments, the projection 422 can be configured to seat against or near a punctal opening when the second portion 406 of the plug body 402 is positioned within the associated canalicular lumen, such as for inhibiting or preventing the punctum plug 400 from passing completely within the canalicular lumen, for providing tactile or visual feedback information to an implanting user (e.g., as to whether the plug is fully implanted), or for removing the punctum plug 400 from an implant position. In an embodiment, the projection 422 includes a portion having a diameter of about 0.5-2.0 mm to prevent the punctum plug 400 from passing down into the canaliculus.

FIG. 4B illustrates an example embodiment of a cross-sectional view of a punctum plug 400 taken along a line parallel to a longitudinal axis of the plug, such as along line 4B-4B of FIG. 4A. As shown in FIG. 4B, the punctum plug 400 comprises a plug body 402, including first 404 and second 406 portions, which is sized and shaped for at least partial insertion into a lacrimal punctum. The first portion 404 is formed from a polymer and includes a first diameter 408. The second portion 406 is also formed from a polymer and includes a base member 412 (e.g., mandrel or spine) having a second diameter 410, which is less than the first diameter 408. In an embodiment, the base member 412 is at least about one-third the total length of the plug body 402. In an embodiment, the base member 412 is at least about one-half the total length of the plug body 402. In the embodiment shown, the plug body 402 also includes an integral feedback or other projection 422, such as a projection extending laterally at least partially from or around a proximal end 418 of the first plug body portion 404.

In various embodiments, the plug body 402 can be molded or otherwise formed using an elastic material, such as silicone, polyurethane or other urethane-based material, or combinations thereof. In an embodiment, one or both of the first 404 and second 406 portions include a urethane-based material. In an embodiment, one or both of the first 404 and second 406 portions include a silicone-based material, such as 4840® or PurSil®. In an embodiment, one or both of the first 404 and second 406 portions include a copolymer material, such as polyurethane/silicone, urethane/carbonate, silicone/polyethylene glycol (PEG) or silicone/2hydroxyethyl methacrylate (HEMA). In various embodiments, the plug body 402 is configured to be non-absorbable in situ and is sufficiently strong to address issues of cutting strength (e.g., during insertion and removal of the punctum plug 400) and dimensional stability.

An expandable retention member 414, such as a swellable material, can be bonded or otherwise coupled over the base member 412 such that it envelops, at least in part, a portion of the base member 412. As the expandable retention member absorbs or otherwise retains lacrimal fluid, such as upon insertion into a lacrimal punctum, its size increases and its shape may change thereby urging itself against and slightly biasing a wall of the associated canaliculus. In various embodiments, the expandable retention member 414 can be molded or otherwise formed using a swellable material. In an embodiment, the expandable retention member 414 includes a polyurethane hydrogel, such as TG-2000®, TG-500®, or other urethane-based hydrogel. In an embodiment, the expandable retention member 414 includes a thermoset polymer, which may be configured to swell anisotropically. In an embodiment, the expandable retention member 414 includes a gel, which does not maintain its shape upon expansion, but rather conforms to fit the shape of a canaliculus lumen wall or other surrounding structure.

In some embodiments, the punctum plug 400 includes a base member 412 including polyurethane or other urethane-based material and an expandable retention member 414 including a polyurethane or other urethane-based swellable material. In an embodiments, a polyurethane hydrogel is coupled directly to an outer surface, such as a plasma-treated outer surface, of the base member 412.

In some embodiments, the punctum plug 400 includes an intermediate member 450 positioned between a portion of the plug body 402, such as the base member 412, and a portion of the expandable retention member 414. The intermediate member 450 can include a material configured to absorb, when implanted, a greater amount of lacrimal fluid than the polymer of the base member 412 but less lacrimal fluid than the swellable polymer of the expandable retention member 414. The intermediate member 450 can provide the punctum plug 400 with integrity, such as between a substantially non-swelling polymer of the plug body 402 and a swelling polymer of the expandable retention member 414. For instance, when the polymer of the expandable retention member 414 swells upon exposure to moisture, it is possible that the expanding polymer will, in the absence of the intermediate member 450, swell away from the underlying, non-swelling polymer of the base member 412. In an embodiment, the intermediate member 450 includes PurSil® and is dip or otherwise coated onto an outer surface of the base member 412. In an embodiment, the intermediate member 450 includes a polyurethane configured to absorb about 10% to about 500% water, such as Tecophilic® urethanes or Tecophilic® solution grade urethanes. Further discussion regarding the use of an intermediate member 450 positioned between a portion of a first polymer material and a portion of a second polymer material, typically different than the first polymer material, can be found in commonly-owned Sim et al., U.S. Application Ser. No. 61/049,329 (filed Apr. 30, 2008 and entitled Composite Lacrimal Insert), which is herein incorporated by reference in its entirety.

In certain embodiments, the plug body 402 can include a cavity 416 disposed near the proximal end 418 of the first portion 404. In an embodiment, the first cavity 416 extends inward about 2 millimeters or less from the proximal end 418, and houses a first therapeutic and/or comforting agent supply 420 to provide a sustained release to an eye. In an embodiment, the first cavity 416 extends through the plug body 402, and houses a first agent-releasing supply 420. In various embodiments, the agent supply 420 stores and slowly dispenses an agent to one or both of the eye or the nasolacrimal system as they are leached out, for example, by tear film fluid or other lacrimal fluid. In an embodiment, the agent supply 420 includes a plurality of therapeutic and/or comforting agent inclusions 452, which can be distributed in a matrix 454. In an embodiment, the inclusions 452 comprise a concentrated form of a therapeutic agent (e.g., a crystalline agent form). In an embodiment, the matrix 454 comprises a silicone matrix or the like, and the distribution of inclusions 452 within the matrix are homogeneous or non-homogeneous. In an embodiment, the agent inclusions 452 include droplets of oil, such as Latanoprost oil. In still another embodiment, the agent inclusions 452 include solid particles, such as Bimatoprost particles in crystalline form. The inclusions can be of many sizes and shapes. For instance, the inclusions can include microparticles having dimensions on the order of about 1 micrometer to about 100 micrometers.

In the embodiment shown, the agent supply 420 includes a sheath body 456 disposed over at least a portion thereof such as to define at least one exposed surface 458 of the supply. In an embodiment, the sheath body 456 comprises polyimide. The exposed surface 458 can be located at or near the proximal end 418 of the plug body 402 such as to contact a tear or a tear film fluid and release the therapeutic and/or comforting agent at one or more therapeutic levels over a sustained time period when the punctum plug 400 is inserted into a lacrimal punctum.

In certain embodiments, the expandable retention member can include a second agent-releasing supply 460 to provide a sustained agent release to one or both of a wall of a lacrimal canaliculus or a nasolacrimal system. The agent supply 460 can be configured to store and slowly dispense an agent after contact with lacrimal fluid within a lacrimal canaliculus. In an embodiment, the agent included in the expandable retention member can comprise medicaments, comforting agents, therapeutic agents, or antimicrobials (e.g., silver).

### Making the Implant:

Particular methods useful for making the implants described herein are described in the above-identified patent documents, the disclosures of which are incorporated herein by reference in their entirety.

In various embodiments, the punctum plugs are made by injection molding. In embodiments comprising multiple polymers, a method of forming the inventive implants, comprise injection molding a first portion, a second portion, or both, using respectively a melt of a first polymer, a second polymer, or melts of both polymers, is provided. Polyurethane polymers and copolymers are adapted for melt processing, thus avoiding both the added complexity of solvent casting technology, the cost of dealing with the necessary solvents, and the possibility of residual solvents in the polymeric materials of the plug. If a third polymer is present, it can also be incorporated into the plug as a melt. The plug can also be made by processes including insert overmolding where the plug body can be molded out of one material and once completed is placed into a second mold where the next material is injected around the part; multi-component molding where there is simultaneous injection of multiple materials into a mold either through the same injection nozzle or separate nozzles; multi-shot molding, where there is sequential injection of separate materials into different locations of the mold; and extrusion of a hydrogel sleeve which is then bonded (via adhesive or melt bonded) to a molded plug body.

The comforting agent and/or therapeutic agent can be mixed directly into the plug material, such as by mechanical mixing, shearing, melt mixing, or ultrasound mixing. In some embodiments, the comforting agent and/or therapeutic agent is mixed directly into uncured silicone, then placed into a mold and heat cured. In some embodiments, the agent is infused into a silicone plug body during the molding/curing process. The agent can be chemically incorporated into the plug material by impregnating the plug using solvents or chemically bonding the comforting agent and/or therapeutic agent. In some embodiments, a molded device can be placed under pressure in a solution of a comforting agent and/or therapeutic agent that is soluble in dichloromethane.

The molding process can form excess flash material, sharp edges, or other irregularities in the molded article's structure. Implantable articles such as punctum plugs, for instance, are placed in direct contact with ocular bodily tissues. The tearing or abrading of such tissues by rough article surfaces or edges can rupture one or more blood vessels, or irritate or cause other tissue trauma. Even small article irregularities can irritate delicate eye tissues. Optionally, the outer surface portion of the plug body can be formed, or surface treated to be, generally smooth to inhibit bacteria from attaching to the punctum plug and incubating. The generally smooth outer surface can also prevent damage to the inner lining of the receiving anatomical tissue, such as a lacrimal punctum or the associated lacrimal canaliculus, during implantation. As further discussed in commonly-owned U.S. Patent Application to Rapacki, Attorney Docket No. 2755.036PV2, titled “SURFACE TREATED IMPLANTABLE ARTICLES AND RELATED METHODS,” filed Jun. 24, 2008, which is herein incorporated by reference in its entirety, the outer surface of the plug body, for instance, can be improved via a polishing procedure using dichloride methane or other suitable media in conjunction with a tumbling process.

The comforting agent and/or therapeutic agent can be incorporated into a thin coating on the surface of the plug body.

Comforting and/or therapeutic agent cores as described above may be fabricated with different cross sectional sizes of 0.006 inches, 0.012 inches, and 0.025 inches. Agent concentrations in the core may be 5%, 10%, 20%, 30% in a silicone matrix. These cores can be made with a syringe tube and cartridge assembly, mixing an agent with silicone, and injecting the mixture into a polyimide tube which is cut to desired lengths and sealed. The length of the cores can be approximately 0.80 to 0.95 mm, which for a diameter of 0.012 inches (0.32 mm) corresponds to total agent content in the cores of approximately 3.5 micrograms, 7 micrograms, 14 micrograms and 21 micrograms for concentrations of 5%, 10%, 20% and 30%, respectively.

Syringe Tube and Cartridge Assembly: 1. Polyimide tubing of various diameters (for example 0.006 inches, 0.0125 inches and 0.025 inches) can be cut to 15 cm length. 2. The polyimide tubes can be inserted into a Syringe Adapter. 3. The polyimide tube can be adhesive bonded into luer adapter (Loctite®, low viscosity UV cure). 4. The end of the assembly can then be trimmed. 5. The cartridge assembly can be cleaned using distilled water and then with methanol and dried in oven at 60.degree. C.

The comforting and/or therapeutic agent can be mixed with silicone. The agent may be provided as a 1% solution in methylacetate. The appropriate amount of solution can be placed into a dish and using a nitrogen stream, the solution can be evaporated until only the agent remains. The dish with the agent oil can be placed under vacuum for 30 minutes. This agent can then be combined with silicone, with three different concentrations of agent (5%, 10% and 20%) in silicone Nusil 6385 being injected into tubing of different diameters (0.006 in, 0.012 in and 0.025 inches) to generate 3×3 matrixes. The percent of agent to silicone is determined by the total weight of the drug matrix. Calculation: Weight of agent/(weight of agent+weight of silicone)×100=percent drug.

The tube can then be injected: 1. The cartridge and polyimide tubes assembly can be inserted into a 1 ml syringe. 2. One drop of catalyst (MED-6385 Curing Agent) can be added in the syringe. 3. Excess catalyst can be forced out of the polyimide tube with clean air. 4. The syringe can then be filled with silicone agent matrix. 5. The tube can then be injected with agent matrix until the tube is filled or the syringe plunger becomes too difficult to push. 6. The distal end of the polyimide tube can be closed off and pressure can be maintained until the silicone begins to solidify. 7. Allow to cure at room temperature for 12 hours. 8. Place under vacuum for 30 minutes. 9. The tube can then be place in the correct size trim fixture (prepared in house to hold different size tubing) and agent inserts can be cut to length (0.80-0.95 mm).

Release of Comforting and/or Therapeutic Agents at Effective Levels:

The rate of release of comforting and/or therapeutic agents can be related to the concentration of agent dissolved in the agent core. In some embodiments, the core comprises non-therapeutic agents that are selected to provide a desired solubility of the comforting and/or therapeutic agent in the drug core. The non-therapeutic agent of the core can comprise polymers as described herein, and additives. A polymer of the core can be selected to provide the desired solubility of the agent in the matrix. For example, the core can comprise hydrogel that may promote solubility of hydrophilic agent. In some embodiments, functional groups can be added to the polymer to provide the desired solubility of the agent in the matrix. For example, functional groups can be attached to silicone polymer.

Additives may be used to control the concentration of the comforting and/or therapeutic agent by increasing or decreasing solubility of the agent in the core so as to control the release kinetics of the agent. The solubility may be controlled by providing appropriate molecules or substances that increase or decrease the content of agent in the matrix. The agent content may be related to the hydrophobic or hydrophilic properties of the matrix and agent. For example, surfactants and salts can be added to the matrix and may increase the content of hydrophobic agent in the matrix. In addition, oils and hydrophobic molecules can be added to the matrix and may increase the solubility of hydrophobic agent in the matrix.

Instead of or in addition to controlling the rate of migration based on the concentration of comforting and/or therapeutic agent dissolved in the matrix, the surface area of the core can also be controlled to attain the desired rate of agent migration from the core to the target site. For example, a larger exposed surface area of the core will increase the rate of migration of the treatment agent from the core to the target site, and a smaller exposed surface area of the core will decrease the rate of migration of the agent from the core to the target site. The exposed surface area of the core can be increased in any number of ways, for example by any of castellation of the exposed surface, a porous surface having exposed channels connected with the tear or tear film, indentation of the exposed surface, protrusion of the exposed surface. The exposed surface can be made porous by the addition of salts that dissolve and leave a porous cavity once the salt dissolves. Hydrogels may also be used, and can swell in size to provide a larger exposed surface area. Such hydrogels can also be made porous to further increase the rate of migration of the comforting and/or therapeutic agent.

Further, an implant may be used that includes the ability to release two or more agents in combination. For example, in the case of glaucoma treatment, it may be desirable to treat a patient with multiple prostaglandins or a prostaglandin and a cholinergic agent or an adrenergic antagonist (beta blocker), such as Alphagan®, or latanoprost and a carbonic anhydrase inhibitor.

In addition, drug impregnated meshes may be used or layering of biostable polymers. Certain polymer processes may be used to incorporate comforting or therapeutic agent into the devices of the present subject matter, such as so-called “self-delivering drugs” or PolymerDrugs (Polymerix Corporation, Piscataway, N.J.) are designed to degrade only into therapeutically useful compounds and physiologically inert linker molecules. Such delivery polymers may be employed in the devices of the present subject matter to provide a release rate that is equal to the rate of polymer erosion and degradation and is constant throughout the course of therapy. Such delivery polymers may be used as device coatings or in the form of microspheres for a drug depot injectable (such as a reservoir of the present subject matter). A further polymer delivery technology may also be configured to the devices of the present subject matter.

In specific embodiments, the core matrix comprises a solid material, for example silicone, that encapsulates inclusions of the comforting or therapeutic agent. The agent comprises molecules which are very insoluble in water and slightly soluble in the encapsulating core matrix. The inclusions encapsulated by the core can be micro-particles having dimensions from about 1 micrometer to about 100 micrometers across. The agent inclusions can comprise droplets of therapeutic agent oil, for example latanoprost oil. The inclusions can dissolve into the solid core matrix and substantially saturate the core matrix with the agent, for example dissolution of latanoprost oil into the solid core matrix. The agent dissolved in the core matrix is transported, often by diffusion, from the exposed surface of the core into the tear film. As the core is substantially saturated with the agent, in many embodiments the rate limiting step of agent delivery is transport of the agent from the surface of the core matrix exposed to the tear film. As the core matrix is substantially saturated with the agent, gradients in concentration within the matrix are minimal and do not contribute significantly to the rate of agent delivery. As surface area of the core exposed to the tear film is nearly constant, the rate of agent transport from the core into the tear film can be substantially constant. It has been found that the solubility of the agent in water and molecular weight of the agent can affect transport of the agent from the solid matrix to the tear. In many embodiments, the comforting or therapeutic agent is nearly insoluble in water and has a solubility in water of about 0.03% to 0.002% by weight and a molecular weight from about 400 grams/mol. to about 1200 grams/mol.

In many embodiments the comforting or therapeutic agent has a very low solubility in water, for example from about 0.03% by weight to about 0.002% by weight, a molecular weight from about 400 grams per mole (g/mol) to about 1200 g/mol, and is readily soluble in an organic solvent. For example, latanoprost is a liquid oil at room temperature, and has an aqueous solubility of 50 micrograms/mL in water at 25 degrees C., or about 0.005% by weight and a M.W. of 432.6 g/mol.

It has been found that naturally occurring surfactants in the tear film, for example surfactant D and phospholipids, may effect transport of the drug dissolved in the solid matrix from the core to the tear film. The core can be configured in response to the surfactant in the tear film to provide sustained delivery of agent into the tear film at therapeutic levels. For example, empirical data can be generated from a patient population, for example 10 patients whose tears are collected and analyzed for surfactant content. Elution profiles in the collected tears for an agent that is sparingly soluble in water can also be measured and compared with elution profiles in buffer and surfactant such that an in vitro model of tear surfactant is developed. An in vitro solution with surfactant based on this empirical data can be used to adjust the agent core in response to the surfactant of the tear film.

The cores may also be modified to utilize carrier vehicles such as nanoparticles or microparticles depending on the size of the molecule to be delivered such as latent-reactive nanofiber compositions for composites and nanotextured surfaces (Innovative Surface Technologies, LLC, St. Paul, Minn.), nanostructured porous silicon, known as BioSilicon®, including micron sized particles, membranes, woven fivers or micromachined implant devices (pSividia, Limited, UK) and protein nanocage systems that target selective cells to deliver an agent (Chimeracore).

In many embodiments, the comforting or therapeutic agent insert comprises a thin-walled polyimide tube sheath with a core comprising the agent dispersed in Nusil 6385 (MAF 970), a medical grade solid silicone that serves as the matrix for delivery. The distal end of the insert is sealed with a cured film of solid Loctite 4305 medical grade adhesive. The insert may be placed within the bore of the punctum plug, the Loctite 4305 adhesive does not come into contact with either tissue or the tear film. The inner diameter of the insert can be 0.32 mm; and the length can be 0.95 mm. At least four agent concentrations in the finished product can be employed: cores can comprise 3.5, 7, 14 or 21 micrograms comforting or therapeutic agent, with percent by weight concentrations of 5, 10, 20, or 30% respectively. Assuming an overall elution rate of approximately 100 ng/day, the core comprising 14 micrograms of the agent is configured to deliver agent for approximately at least 100 days, for example 120 days. The overall weight of the core, including the agent, can be about 70 micrograms. The weight of the insert including the polyimide sleeve can be approximately 100 micrograms.

### Methods of Treatment:

The subject matter described herein provides methods to treat ocular conditions including, but not limited to: glaucoma, elevated intraocular pressure (ocular hypertension), dry eye, conjunctivitis, pre- and post-surgical conditions, and irritation/inflammation associated with allergy.

Cyclosporine, employed in the treatment of dry eye, can cause discomfort to the patient. In some embodiments, the punctum plugs described herein are employed to deliver cyclosporine and a comforting agent to the eye to minimize the discomfort. In other embodiments, the punctum plugs described herein are employed in combination with eye drop administration of cyclosporine to relieve and minimize discomfort. In other embodiments, the punctum plugs described herein deliver a comforting agent and no additional therapeutic agents. In other embodiments, the punctum plugs described herein release latanoprost and a comforting agent to treat glaucoma and ocular hypertension.

In some embodiments, the therapeutic agent is released to the eye over a sustained period of time. In an embodiment, the sustained period of time is approximately 90 days. In some embodiments, the method comprises inserting through a punctum an implant having a body and a core so that the core is retained near the punctum. In some embodiments, the method comprises inserting through a punctum an implant having a body impregnated with a therapeutic agent. An exposed surface of the core or impregnated body located near the proximal end of the implant contacts the tear or tear film fluid and the therapeutic agent migrates from the exposed surface to the eye over a sustained period of time while the core and body is at least partially retained within the punctum. In many embodiments, a method of treating an eye with a therapeutic agent is provided, the method comprising inserting through a punctum into a canalicular lumen an implant having an optional retention structure so that the implant body is anchored to a wall of the lumen by the retention structure. The implant releases effective amounts of a therapeutic agent from a core or other agent supply into a tear or tear film fluid of the eye. In some embodiments, the core may be removed from the retention structure while the retention structure remains anchored within the lumen. A replacement core can then be attached to the retention structure while the retention structure remains anchored. At least one exposed surface of the replacement core releases the therapeutic agent at therapeutic levels over a sustained period.

A replacement core can be attached to the retention structure approximately every 90 days to result in continuous release of the therapeutic agent to the eye for a period of time of approximately 180 days, approximately 270 days, approximately 360 days, approximately 450 days, approximately 540 days, approximately 630 days, approximately 720 days, approximately 810 days or approximately 900 days. In some embodiments, a replacement plug can be inserted into the punctum approximately every 90 days to achieve release of the agent to the eye for extended periods of time, including up to about 180 days, about 270 days, about 360 days, about 450 days, about 540 days, about 630 days, about 720 days, about 810 days or about 900 days.

In other embodiments, a method for treating an eye with a comforting or therapeutic agent is provided, the method comprising inserting a core or other implant body at least partially into at least one punctum of the eye. The core may or may not be associated with a separate implant body structure. The core or agent-impregnated implant body provides sustained release delivery of a therapeutic agent at therapeutic levels and a shorter-term release of a comforting agent. In some embodiments, the sustained release delivery of the therapeutic agent continues for up to 90 days.

In many embodiments, a method for treating an eye with a comforting or therapeutic agent is provided, the method comprising inserting a distal end of an implant into at least one punctum of the eye. In some embodiment, a retention structure of the implant can be expanded so as to inhibit expulsion of the implant. The expansion of the retention structure can help to occlude a flow of tear fluid through the punctum. In some embodiments, the implant is configured such that, when implanted, an at least 45 degree angled intersection exists between a first axis, defined by a proximal end of the implant, and a second axis, defined by the distal end of the implant, to inhibit expulsion of the implant. The comforting or therapeutic agent is delivered from a proximal end of the implant to the tear fluid adjacent the eye. Delivery of the comforting or therapeutic agent is inhibited distally of the proximal end.

The methods of the present subject matter provide sustained release of a therapeutic agent. In some embodiments, the agent is released from the implant for at least one week, at least two weeks, at least three weeks, at least four weeks, at least five weeks, at least six weeks, at least seven weeks, at least eight weeks, at least nine weeks, at least ten weeks, at least eleven weeks, at least twelve weeks, at least thirteen weeks, at least fourteen weeks, at least fifteen weeks, or at least sixteen weeks. In an embodiment, the therapeutic agent is released for at least twelve weeks. In another embodiment, the methods of treatment further comprises an adjunctive therapy with a therapeutic agent-delivering eye drop solution, for example, latanoprost (Xalatan®).

The comforting agent can be released from the punctum plug for approximately one day, approximately two days, approximately three days, approximately four days, approximately five days, approximately six days, approximately seven days, approximately eight days, approximately nine days, approximately ten days, approximately eleven days, approximately twelve days, approximately thirteen days, approximately fourteen days, approximately fifteen days, approximately sixteen days, approximately seventeen days, approximately eighteen days, approximately nineteen days, approximately twenty days, approximately twenty-one days, approximately twenty-two days, approximately twenty-three days, approximately twenty-four days, approximately twenty-five days, approximately twenty-six days, approximately twenty-seven days, approximately twenty-eight days, approximately twenty-nine days, or approximately thirty days, after insertion of the punctum plug. The comforting agent can be released from the punctum plug for longer than approximately thirty days after insertion of the punctum plug, including for example approximately five weeks, approximately six weeks, approximately seven weeks, or approximately eight weeks, or longer.

The amount of comforting or therapeutic agent associated with the implant may vary depending on the desired therapeutic benefit and the time during which the device is intended to deliver the therapy. Since the devices of the present subject matter present a variety of shapes, sizes and delivery mechanisms, the amount of agent associated with the device will depend on the particular disease or condition to be treated, and the dosage and duration that is desired to achieve the therapeutic effect. Generally, the amount of comforting or therapeutic agent is at least the amount of agent that, upon release from the device, is effective to achieve the desired physiological or pharmacological local or systemic effects. In embodiments having both comforting and therapeutic agents, the amount of each agent can be the same or different. The rates of release can also be the same or different.

Embodiments of the implants of the present subject matter can be configured to provide delivery of comforting or therapeutic agent at a daily rate that is substantially below the therapeutically effective drop form of treatment so as to provide a large therapeutic range with a wide safety margin. For example, many embodiments treat the eye with therapeutic levels for extended periods that are no more than 5 or 10 percent of the daily drop dosage. In specific embodiments, the quantity can be less than 5% of the recommended drop-administered quantity. Consequently, during an initial bolus or washout period of about one to three days, the implant can elute the therapeutic agent at a rate that is substantially higher than the sustained release levels and well below the daily drop form dosage. For example, with an average sustained release level of 100 ng per day, and an initial release rate of 1000 to 1500 ng per day, the amount of agent initially released is less than the 2500 ng of therapeutic agent that may be present in a drop of therapeutic agent delivered to the eye. This use of sustained release levels substantially below the amount of agent in one or more drops administered daily allows the device to release a therapeutically beneficial amount of agent to achieve the desired therapeutic benefit with a wide safety margin, while avoiding an inadequate or excessive amount of agent at the intended site or region. In many embodiments, the core may elute with an initial elevated level of the comforting or therapeutic agent followed by substantially constant elution of the comforting or therapeutic agent. In many instances, an amount of agent released daily from the core may be below the therapeutic levels and still provide a benefit to the patient. An elevated level of eluted agent can result in a residual amount of agent or residual effect of the agent that is combined with a sub-therapeutic amount of the agent to provide relief to the patient. In embodiments where therapeutic level is about 80 ng per day, the device may deliver about 100 ng per day for an initial delivery period. The extra 20 ng delivered per day can have a beneficial effect when the agent is released at levels below the therapeutic level, for example at 60 ng per day. As the amount of agent delivered can be precisely controlled, an initial elevated dose may not result in complications or adverse events to the patient.

In certain embodiments, the methods of the present subject matter result in a percentage reduction in intraocular pressure of approximately 28%. In some embodiments, the methods result in a percentage reduction in intraocular pressure of approximately 27%, approximately 26%, approximately 25%, approximately 24%, approximately 23%, approximately 22%, approximately 21%, or approximately 20%. In certain embodiments, the methods result in a percentage reduction in intraocular pressure of at least 28%, at least 27%, at least 26%, at least 25%, at least 24%, at least 23%, at least 22%, at least 21%, or at least 20%.

In certain embodiments, the methods of the present subject matter result in a reduction in intraocular pressure from baseline of about 6 mm Hg, about 5 mm Hg, about 4 mm Hg, about 3 mm Hg or about 2 mm Hg. In certain embodiments, the methods result in a reduction in intraocular pressure from baseline of at least 2 mm Hg, at least 3 mm Hg, at least 4 mm Hg, at least 5 mm Hg, or at least 6 mm Hg.

In an embodiment, the implants and methods of the present subject matter provide a 90-day course of treatment. In some embodiments, effective levels of the therapeutic agent are released during the entire course of treatment, while effective levels of the comforting agent are released for a shorter period of time. In a further embodiment, the variability in intraocular pressure over the course of treatment is less than about 1 mm Hg. In other embodiments, the variability in intraocular pressure over the course of treatment is less than about 2 mm Hg. In other embodiments, the variability in intraocular pressure over the course of treatment is less than about 3 mm Hg.

Methods of inserting and removing the implant are known to those of skill in the art. For instance, tools for insertion and removal/extraction of implants are described in commonly-owned U.S. Patent Application No. 60/970,840 (filed Sep. 7, 2007 and entitled Insertion and Extraction Tools for Punctal Implants), the disclosure of which is incorporated herein in its entirety. Generally, for placement, the size of punctal plug to be used may be determined by using suitable magnification or, if provided, using a sizing tool that accompanies the punctal plug. The patient's punctum may be dilated if necessary to fit the punctal plug. A drop of lubricant may be applied if necessary to facilitate placement of the plug into the punctum. Using an appropriate placement instrument, the plug may be inserted into the superior or inferior punctum of the eye. After placement, the cap of the plug may be visible. This process may be repeated for the patient's other eye. For removal of the implant, small surgical forceps may be used to securely grasp the plug at the tube section below the cap. Using a gentle tugging motion the plug may be gently retrieved. The implants described herein may be inserted into the superior punctum, the inferior punctum, or both, and may be inserted into one or both eyes of the subject.

Various methods to assess comfort and efficacy provided by the punctum plugs described herein are known in the art. For example, objective indicia include corneal staining; interpalpebral conjunctival staining; rose bengal staining; fluorescein staining; fluorescein clearance testing; nasal-lacrimal reflex tearing; superficial punctate keratitis measurements; Schirmer tear tests; intraocular pressure measurements, and tear beak-up time. Subjective indicia include the Ocular Surface Disease Index (OSDI); National Eye Institute Vision Functioning Questionnaire (NEI-VFQ); facial expression subjective rating scale; report of symptoms of ocular discomfort, such as stinging/burning, itching, sandiness/grittiness, blurred vision, dryness, light sensitivity, pain or soreness, graded using a 5-point scale ranging from absence of symptoms to always noticing these symptoms; tear film debris; and the use of artificial tears. Another subjective assessment of comfort is based on a 100-point analogue scale. The scale has four descriptor points equidistant from the two extremes at opposite ends of the scale. On the scale, zero (0) represents severe discomfort; 20 represents moderate discomfort; 40 represents mild discomfort; 60 represents moderate awareness of discomfort; 80 represents mild awareness of discomfort; and 100 represents no awareness of discomfort. Assessments can be made at designated time points after insertion of the punctum plugs described herein. These indicia are further described in Sall et al., Opthalmology 107(4):631-9 (2000); Stevenson et al., Opthalmology 107(5):967-74 (2000); Barber et al., Opthalmology 112(10):1790-4 (2005); Vitale et al., Health and Quality of Life Outcomes 2:44 http://www.hqlo.com/content/2/1/44 (2004); and Pflugfelder et al., Cornea 17(1):38-56 (1998), the disclosures of which are incorporated herein by reference in their entirety.

The above Detailed Description includes references to the accompanying drawings, which form a part of the Detailed Description. The drawings show, by way of illustration, specific embodiments in which the present subject matter can be practiced. These embodiments are also referred to herein as “examples.” All publications, patents, and patent documents referred to in this document are incorporated by reference herein in their entirety, as though individually incorporated by reference. In the event of inconsistent usages between this document and those documents so incorporated by reference, the usage in the incorporated reference(s) should be considered supplementary to that of this document; for irreconcilable inconsistencies, the usage in this document controls.

The above description is intended to be illustrative, and not restrictive. For example, the above-described examples (or one or more features thereof) can be used in combination with each other. Other embodiments can be used, such as by one of ordinary skill in the art upon reviewing the above description. Also, in the above Detailed Description, various features can be grouped together to streamline the disclosure. This should not be interpreted as intending that an unclaimed disclosed feature is essential to any claim. Rather, inventive subject matter can lie in less than all features of a particular disclosed embodiment. Thus, the following claims are hereby incorporated into the Detailed Description, with each claim standing on its own as a separate embodiment. The scope of the present subject matter should be determined with reference to the appended claims, along with the full scope of equivalents to which such claims are entitled.

1. (canceled)

2. A punctum plug insertable into a lacrimal punctum for release of one or more comforting agents to an eye, the punctum plug comprising:
a plug body having a proximal end portion adapted to be disposed near the punctal opening and a distal end portion adapted to be disposed within the punctal canal;
wherein the punctum plug provides release of the one or more comforting agents to the eye.

3. The punctum plug of claim 2, wherein at least one of the one or more comforting agents is a demulcent or emollient.

4. (canceled)

5. The punctum plug of claim 2, wherein the one or more comforting agents is embedded in the plug body or coated on the surface of the plug body.

6. (canceled)

7. The punctum plug of claim 2, wherein the one or more comforting agents is infused or mixed into the plug body.

8. (canceled)

9. The punctum plug of claim 2, further comprising an agent core inserted at or near the proximal end portion.

10. The punctum plug of claim 9, wherein the agent core provides release of a therapeutic agent or agents, the one or more comforting agents, or both, to the eye.

11. The punctum plug of claim 9, wherein both the agent core and the plug body provide release of the one or more comforting agents to the eye.

12. (canceled)

13. The punctum plug of claim 2, wherein the one or more comforting agents is selected from the group consisting of polyvinyl alcohol (PVA), polyethylene oxide (PEO), polyvinyl pyrrolidone (PVP), hydroxypropyl methylcellulose (HPMC), carboxy methylcellulose (CMC), glycerin, simethicone, and sodium hyaluronate.

14. The punctum plug of claim 2, wherein the punctum plug further provides release of a therapeutic agent or agents to the eye.

15-16. (canceled)

17. The punctum plug of claim 2, wherein the plug body comprises a silicone, a silicone copolymer, a polyurethane, a polyurethane copolymer, a polyurethane-silicone copolymer, or any combination thereof.

18-20. (canceled)

21. A punctum plug insertable into a lacrimal punctum for release of a therapeutic agent or agents, a comforting agent or agents, or both to an eye, the punctum plug comprising:
a plug body having a proximal end portion adapted to be disposed near the punctal opening and a distal end portion adapted to be disposed within the punctal canal;
an agent core at or near the proximal end portion;
a retention structure at or near the distal end portion; and
wherein the agent core or the plug body has at least one surface that provides release of the therapeutic agent or agents, the comforting agent or agents, or both, to the eye.

22. The plug of claim 21, wherein the agent core or the plug body provides release of the therapeutic agent or agents.

23. (canceled)

24. The plug of claim 21, wherein both the agent core and the plug body provide release of the therapeutic agent or agents.

25. The plug of claim 21, wherein the agent core or the plug body provides release of the comforting agent or agents.

26. (canceled)

27. The plug of claim 21, wherein both the agent core and the plug body provide release of the comforting agent or agents.

28. The plug of claim 21, wherein the agent core or the plug body provides release of both the therapeutic agent or agents and the comforting agent or agents.

29. The plug of claim 21, wherein the agent core and the plug body provide release of both the therapeutic agent or agents and the comforting agent or agents.

30. The punctum plug of claim 21, wherein the comforting agent is a demulcent or emollient.

31. The punctum plug of claim 21, wherein the release of the therapeutic agent or agents is a sustained release.

32. (canceled)

33. The punctum plug of claim 21, wherein the plug body comprises a silicone, a silicone copolymer, a polyurethane, a polyurethane copolymer, a polyurethane-silicone copolymer, or any combination thereof.

34. (canceled)

35. The punctum plug of claim 21, wherein the comforting agent is selected from the group consisting of polyvinyl alcohol (PVA), polyethylene oxide (PEO), polyvinyl pyrrolidone (PVP), hydroxypropyl methylcellulose (HPMC), carboxy methylcellulose (CMC), glycerin, simethicone, and sodium hyaluronate.

36. The punctum plug of claim 21, wherein the comforting agent or agents are coated on the surface of the plug body, agent core, or both.

37. The punctum plug of claim 21, wherein the comforting agent or agents are infused or mixed into the plug body, agent core, or both.

38-42. (canceled)

43. The punctum plug of claim 21, wherein the agent core comprises at least one therapeutic agent inclusion distributed in a solid matrix and at least partially covered by a sheath body to define at least one exposed agent core surface.

44. The punctum plug of claim 43, wherein the solid matrix comprises a mixture of silicone and the therapeutic agent.

45. The punctum plug of claim 43, wherein the solid matrix comprises a mixture of silicone, the therapeutic agent, and the comforting agent.

46-53. (canceled)

54. A method of manufacturing a punctum plug insertable into a lacrimal punctum for release of one or more comforting agents to an eye, the method comprising:
forming a plug body having a proximal end portion adapted to be disposed near the punctal opening and a distal end portion adapted to be disposed within the punctal canal;
wherein the punctum plug provides release of the one or more comforting agents to the eye.

55. The method of claim 54, wherein the comforting agent is a demulcent or emollient.

56. The method of claim 54, wherein forming the plug body includes:
mixing the comforting agent into a silicone;
placing the mixture into a mold; and
heat curing the mixture.

57. (canceled)

58. A method to treat a subject having an eye disorder, comprising:
inserting a punctum plug into at least one lacrimal punctum of the subject, the punctum plug comprising,
a plug body having a proximal end portion adapted to be disposed near the punctal opening and a distal end portion adapted to be disposed within the punctal canal;
wherein the plug body comprises an agent core at or near the proximal end portion and a retention structure at or near the distal end portion;
wherein the agent core comprises a therapeutic agent or agents and has at least one surface providing release of the therapeutic agent or agents to the eye; and
wherein the agent core, plug body or both provide release of a comforting agent or agents to the eye.

59. The method of claim 58, wherein the comforting agent is a demulcent or emollient.

60. The method of claim 58, wherein the comforting agent or agents are embedded in the agent core.

61. The method of claim 58, wherein the comforting agent or agents are embedded in both the agent core and plug body.

62. The method of claim 58, wherein the comforting agent or agents are coated on the surface of the plug body, agent core, or both.

63. The method of claim 58, wherein the comforting agent or agents are infused or mixed into the plug body, agent core, or both.

64-67. (canceled)

68. The method of claim 58, wherein the subject feels less discomfort compared to a subject who is treated with a punctum plug lacking the comforting agent or agents.

69-71. (canceled)

72. The method of claim 58, wherein the eye disorder is glaucoma or dry eye.

73. (canceled)

74. The method of claim 72, further comprising administering an anti-glaucoma medication selected from the group consisting of adrenergic agonists, adrenergic antagonists, carbonic anhydrase inhibitors, parasympathomimetics, prostaglandins and hypotensive lipids, and combinations thereof.

75-77. (canceled)

78. The method of claim 58, comprising replacing the inserted punctum plug with a second punctum plug having the same, lower or higher dosage of the agent following an interval of time.

79-80. (canceled)

81. The method of claim 78, wherein the step of replacing the punctum plug is repeated until the subject no longer requires treatment.

82. The method of claim 58, wherein the comforting agent is selected from the group consisting of polyvinyl alcohol (PVA), polyethylene oxide (PEO), polyvinyl pyrrolidone (PVP), hydroxypropyl methylcellulose (HPMC), carboxy methylcellulose (CMC), glycerin, simethicone, and sodium hyaluronate.

83. The method of claim 58, wherein the comforting agent is released for at least one week, at least two weeks, or at least one month.

84. (canceled)
